(19) United States
(12) Patent Application Publication
(10) Pub. No.: US 2023/0295749 A1
(43) Pub. Date: Sep. 21, 2023

(54) METHODS AND SYSTEMS FOR DETECTING AND DISCRIMINATING BETWEEN VIRAL VARIANTS

(71) Applicant: Rutgers, The State University of New Jersey, New Brunswick, NJ (US)

(72) Inventors: David Alland, Bernardsville, NJ (US); Padmapriya P. Banada, Edison, NJ (US); Salvatore A.E. Marras, Roselle Park, NJ (US)

(73) Assignee: Rutgers, The State University of New Jersey, New Brunswick, NJ (US)

(21) Appl. No.: 18/154,427

(22) Filed: Jan. 13, 2023

Related U.S. Application Data
(60) Provisional application No. 63/266,818, filed on Jan. 14, 2022.

(51) Int. Cl. C12Q 1/70

(52) U.S. Cl. CPC C12Q 1/701; C12Q 2600/156; C12Q 2600/16

(57) ABSTRACT

This disclosure provides an improved assay capable of identifying and distinguishing viral variants, such as SARS-CoV-2 variants and subvariants, including Alpha, Beta, Gamma, Delta, and Omicron, with high specificity and sensitivity. The disclosed assay can be used for real-time monitoring of viral variants (e.g., SARS-CoV-2 variants) spread without the need for whole-genome sequencing on all samples.

### CROSS-REFERENCE TO RELATED APPLICATIONS

This application claims priority under 35 U.S.C. § 119(e) to U.S. Provisional Patent Application No. 63/266,818, filed Jan. 14, 2022. The foregoing application is incorporated by reference herein in its entirety.

### STATEMENT REGARDING FEDERALLY FUNDED RESEARCH

This invention was made with government support under Grant No. R01AI131617 awarded by The National Institute of Allergy and Infectious Diseases. The government has certain rights in the invention.

### REFERENCE TO A SEQUENCE LISTING

This application incorporates by reference the Sequence Listing submitted in Computer Readable Form as file Seq_Listing_09674700476.xml, created on Apr. 17, 2023 and containing 18,174 bytes.

### FIELD OF THE INVENTION

This invention relates to methods and uses for detecting and discriminating between viral variants, such as SARS-CoV-2 variants.

### BACKGROUND OF THE INVENTION

The global emergence of SARS-CoV-2 variants of concern (VOC), B.1.1.7 (201, Alpha), B.1.351 (20H, Beta), P.1 (20J, Gamma), B.1.617.2 (21A, Delta), and B.1.1.529 (21K, Omicron) have been responsible for a series of surges in reported COVID-19 cases. The Omicron variant, first identified as a VOC by the WHO in November 2021 and is now comprised of several sub lineages. As of Aug. 25, 2022, Omicron BA.5 has dominated the United States accounting for 84.2% of the COVID-19 cases, followed by Omicron BA.4 at 13.3%, Omicron BA.2.12.1 at 1.2%, Omicron BA.2 at 0.8%, and Omicron BA.2.75 at 0.4%. Several studies have indicated that these variants are more transmissible and possibly more virulent than other SARS-CoV-2 strains, and the CDC and others have indicated that the Omicron subvariant spreads even more rapidly than Delta. SARS-CoV-2 variants, may confer resistance to therapeutics and decreased vaccine efficacy due to the presence of key mutations in the spike protein. Although the United States has recently increased its capacity to track variants by genome sequencing, only 10% of COVID-19 cases are currently being sequenced, and the results of these efforts are often delayed, threatening their utility in preventing further spread or providing real-time therapeutic guidance. A more rapid approach to screen for potential Omicron variants takes advantage of specific RT-PCR COVID-19 tests, which fail to detect S-gene target sequences in Omicron variants. Samples that are negative for the spike target but positive for another SARS-CoV-2 sequence identify presumptive Omicron infections, which are then confirmed DNA sequencing. However, this type of test only identifies Omicron variants, and it still requires sequence confirmation and the associated time delay.

Thus, there is a pressing need for improved methods for detecting and discriminating viral variants, such as SARS-CoV-2 variants.

### SUMMARY OF THE INVENTION

This disclosure addresses the need mentioned above in a number of aspects. In one aspect, this disclosure provides a versatile and specific melting temperature (Tm)-based assay for detecting and discriminating variants of a virus, such as SARS-CoV-2 variants of concern (VOC), including Delta and Omicron variants.

In one aspect, this disclosure provides a method for identifying a variant/subvariant of a virus, such as SARS-CoV-2. In some embodiments, the method comprises: (a) amplifying a viral nucleic acid of a variant of a virus in a sample with one or more primer pairs to obtain one or more amplicons, wherein each of the one or more primer pairs comprises a forward primer and a reverse primer, wherein the one or more primer pairs each specific for a target region of the viral nucleic acid, and wherein the one or more amplicons respectively correspond to one or more target regions of the viral nucleic acid; (b) contacting the one or more amplicons with one or more probes under a condition conducive to a hybridization reaction to form one or more probe-amp/icon hybrids; (c) determining a melting temperature (Tm) of each of the one or more probe-amplicon hybrids; (d) determining a difference between the melting temperature of each of the one or more probe-amplicon hybrids and a reference melting temperature corresponding to the same probe-amplicon hybrid; (c) determining an aggregated difference of melting temperatures of the one or more probe-amplicon hybrids based on the difference between the melting temperature of each of the one or more probe-amplicon hybrids and the reference melting temperature corresponding to the same probe-amplicon hybrid; and (f) identifying the variant of the virus in the sample as a candidate variant, if the aggregated difference of the melting temperatures of the one or more probe-amplicon hybrids is identical to a reference aggregated difference of the candidate variant or if a difference between the aggregated difference of the melting temperatures of the one or more probe-amplicon hybrids and the reference aggregated difference of the candidate variant is less than a threshold value.

In some embodiments, the variant is a SARS-CoV-2 variant. In some embodiments, the SARS-CoV-2 variant is selected from B.1.1.7 (201, Alpha), B.1.351 (20H, Beta), P.1 (20J, Gamma), B.1.617.2 (21A, Delta), and B.1.1.529 (21K, Omicron).

In some embodiments, the step of amplifying is performed by a polymerase chain reaction (PCR).

In some embodiments, the sample comprises a viral genomic RNA or fragment thereof; and wherein prior to the step of amplifying, the method comprises a reverse transcription step by a reverse transcription-polymerase chain reaction (RT-PCR). In some embodiments, the method comprises extracting the viral genomic RNA or fragment thereof from the sample.

In some embodiments, the step of amplifying, the reverse transcription step, and the step of contacting are performed in a single reaction mixture. In some embodiments, the step of amplifying for each of the one or more primer pairs is performed in separate reaction mixtures.

In some embodiments, at least one of the one or more target regions comprises one or more mutations characteristic of the variant of the virus. In some embodiments, the one or more target regions comprise a codon of L452 or a substitution thereof, a codon of E484 or a substitution thereof, a codon of N501 or a substitution thereof; or a combination thereof. In some embodiments, the one or more target regions comprise a codon of L452 or a substitution of L452Q or L452R, codon of E484 or a substitution of E484K or E484A, a codon of N501 or a substitution of N501Y, or a combination thereof.

In some embodiments, the one or more primer pairs comprise a first primer pair capable of hybridizing to a first target region of the viral nucleic acid, a second primer pair capable of hybridizing to a second target region of the viral nucleic acid, and a third primer pair capable of hybridizing to a third target region of the viral nucleic acid.

In some embodiments, the first target region of the viral nucleic acid comprises a codon of L452 or a substitution of L452Q or L452R, the second target region of the viral nucleic acid comprises a codon of E484 or a substitution of E484K or E484A, and the third target region of the viral nucleic acid comprises a codon of N501 or a substitution of N501Y.

In some embodiments, the one or more amplicons comprise a first amplicon comprising the first target region, a second amplicon comprising the second target region, and a third amplicon comprising the third target region.

In some embodiments, the one or more probes comprise a first probe or a second probe capable of hybridizing to the first amplicon, a third probe or a fourth probe capable of hybridizing to the second amplicon, and a fifth probe or a sixth probe capable of hybridizing to the third amplicon.

In some embodiments, the first primer pairs comprise respective nucleotide sequences of SEQ NOs: 1-2, or comprise the respective nucleotide sequences having at least 90% sequence identity with the nucleotide sequences of SEQ ID NOs: 1-2; the second primer pairs comprise respective nucleotide sequences of SEQ ID NOs: 5-6 or SEQ ID NOs: 7-8; or comprise the respective nucleotide sequences having at least 90% sequence identity with the nucleotide sequences of SEQ ID NOs: 5-6 or SEQ ID NOs: 7-8; and/or the third primer pairs comprise respective nucleotide sequences of SEQ ID NOs: 11-12, or comprise the respective nucleotide sequences having at least 90% sequence identity with the nucleotide sequences of SEQ ID NOs: 11-12.

In some embodiments, the first probe comprises the nucleotide sequence of SEQ ID NO: 3, or comprises a nucleotide sequence having at least 90% sequence identity with the nucleotide sequence of SEQ ID NO: 3; and/or the second probe comprises the nucleotide sequence of SEQ ID NO: 4, or comprises a nucleotide sequence having at least 90% sequence identity with the nucleotide sequence of SEQ ID NO: 4.

In some embodiments, the third probe comprises the nucleotide sequence of SEQ ID NO: 9, or comprises a nucleotide sequence having at least 90% sequence identity with the nucleotide sequence of SEQ ID NO: 9; and/or the fourth probe comprises the nucleotide sequence of SEQ ID NO: 10, or comprises a nucleotide sequence having at least 90% sequence identity with the nucleotide sequence of SEQ ID NO: 10.

In some embodiments, the fifth probe comprises the nucleotide sequence of SEQ ID NO: 13, or comprises a nucleotide sequence having at least 90% sequence identity with the nucleotide sequence of SEQ ID NO: 13; and/or the sixth probe comprises the nucleotide sequence of SEQ ID NO: 14, or comprises a nucleotide sequence having at least 90% sequence identity with the nucleotide sequence of SEQ ID NO: 14.

In some embodiments, the aggregated difference of the melting temperature is determined based on an equation set forth as follows:
aggregated difference=\sqrt{Σᵢ₌₀ⁿ(Tmᵢ−Tmᵢ^{ref})²}, wherein Tmᵢ is a melting temperature determined for probe i, Tmᵢ^{ref} is a reference melting temperature determined for probe i, and n is is an integer that denotes the number of probes.

In some embodiments, the one or more probes comprise one or more labels. In some embodiments, the one or more labels comprise at least one of a fluorophore and a quencher. In some embodiments, the fluorophore is selected from fluorescein, cyanine 3, cyanine 5, TexasRed, and TAMRA. In some embodiments, the quencher is selected from BHQ1, BHQ2, and DABCYL.

Also within the scope of this disclosure is a system for identifying a variant/subvariant of a virus, such as SARS-CoV-2. In some embodiments, the system is configured to: (i) amplify a viral nucleic acid of a variant of a virus in a sample with one or more primer pairs to obtain one or more amplicons, wherein each of the one or more primer pairs comprises a forward primer and a reverse primer, wherein the one or more primer pairs each specific for a target region of the viral nucleic acid, and wherein the one or more amplicons respectively correspond to one or more target regions of the viral nucleic acid; (ii) contact the one or more amplicons with one or more probes under a condition conducive to a hybridization reaction to form one or more probe-amplicon hybrids; (iii) determine a melting temperature (Tm) of each of the one or more probe-amplicon hybrids; (iv) determine a difference between the melting temperature of each of the one or more probe-amplicon hybrids and a reference melting temperature corresponding to the same probe-amplicon hybrid; (v) determine an aggregated difference of melting temperatures of the one or more probe-amplicon hybrids based on the difference between the melting temperature of each of the one or more probe-amplicon hybrids and the reference melting temperature corresponding to the same probe-amplicon hybrid; and (vi) identify the variant of the virus in the sample as a candidate variant, if the aggregated difference of the melting temperatures of the one or more probe-amplicon hybrids is identical to a reference aggregated difference of the candidate variant or if a difference between the aggregated difference of the melting temperatures of the one or more probe-amplicon hybrids and the reference aggregated difference of the candidate variant is less than a threshold value.

In another aspect, this disclosure also provides an isolated nucleic acid, such as a primer or a probe, for identifying a SARS-CoV-2 variant. In some embodiments, the isolated nucleic acid comprises a nucleotide sequence of SEQ ID NO: 1-14 or comprises a nucleotide sequence having at least 90% sequence identity with a nucleotide sequence of SEQ ID NO: 1-14.

In some embodiments, the isolated nucleic acid comprises a nucleotide sequence of SEQ ID NO: 5, 6, 9, 10, 13, and 14, or comprises a nucleotide sequence having at least 90% sequence identity with a nucleotide sequence of SEQ ID NO: 5, 6, 9, 10, 13, and 14.

In yet another aspect, this disclosure provides a kit for identifying a SARS-CoV-2 variant. In some embodiments, the kit comprises the isolated nucleic acid as disclosed herein.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows an example process for one-step RT-PCR followed by melt analysis.

FIGS. 2A and 2B show SMB 484 assay evaluation with variants containing a mutation at the 484 codon region. The assay probes were tested against short synthetic probes (FIG. 2A) and with amplified WT and B.1.351 (Beta) RNA or synthetic amplified target from Omicron (B.1.529) specific sequence (FIG. 2B).

FIGS. 3A and 3B show a sloppy molecular beacon (SMB) Tm profile of positive clinical nasopharyngeal (NP) samples tested using the SMB-501 and SMB-484 Tm assay. Tm signatures consisting of the Tm values for both the WT probe and MT probe are shown for the SMB-501 assay (FIG. 3A) and the SMB-484 assay (FIG. 3B). Ref WT indicates the Tm profile of the reference, WT SARS-CoV-2 strain. Ref MT indicates the TM profile of the reference MT SARS-CoV-2 B.1.1.7 strain (FIG. 3A) and B.351 (FIG. 3B). SMBP1-SMBP46 indicate that Tm profiles of the 46 COVID positive clinical samples tested with the SMB-501 assay and the 26 COVID positive samples tested by the SMB-484 assay. Error bars show +/−one standard deviation.

FIGS. 4A, 4B, 4C, 4D, 4E, 4F, 4G, and 4H show variant of concern (VOC) detection using the SARS-COV-2 SMB-VOC assay. Correlation plots show grouping and classification of patient specimens (N=90) tested in Roche LC480 instrument using SMB-501, SMB-484, and SMB-452 assays (FIG. 4A). Identification Tm signature specific for the wild type (WT, FIG. 4B); Alpha (FIG. 4C); Delta (FIG. 4D); Omicron BA.1 (FIG. 4E); Omicron BA.2 (FIG. 4F); Omicron BA.2.12.1 (FIG. 4G); and Omicron BA.4/5 (FIG. 4H) are highlighted and indicated by arrows. + indicates the number of mutations in the codon against the VOC.

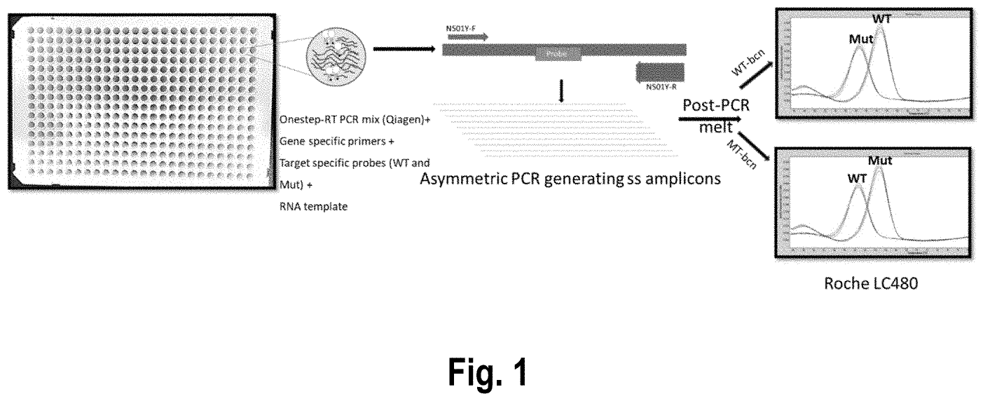

### DETAILED DESCRIPTION OF THE INVENTION

The worrisome emergence of highly transmissible and SARS-CoV-2 variants of concern (VOC) that are vaccine resistant, such as Delta (B.1.617.2) and Omicron (B.1.1.529, e.g., BA.2, BA.4, and BA.5), emphasizes the need for efficient and high throughput tests for SARS-CoV-2 VOC surveillance. This disclosure provides an improved assay capable of identifying and distinguishing viral variants, such as SARS-CoV-2 variants, including Alpha, Beta, Gamma, Delta, and Omicron, with high specificity and sensitivity. The disclosed assay can be used for real-time monitoring of viral variants (e.g., SARS-CoV-2 variants) spread without the need for whole-genome sequencing on all samples.

Accordingly, in one aspect, this disclosure provides a versatile, and specific melting temperature-based assay for detecting and discriminating variants of a virus, such as SARS-CoV-2 variants of concern (VOC), including Alpha, Beta, Gamma, Delta, and Omicron variants.

In one aspect, this disclosure provides a method for identifying a variant/subvariant of a virus, such as SARS-CoV-2. In some embodiments, the method may include: (a) amplifying a viral nucleic acid of a variant of a virus in a sample with one or more primer pairs to obtain one or more amplicons, wherein each of the one or more primer pairs may include a forward primer and a reverse primer, wherein the one or more primer pairs each specific for a target region of the viral nucleic acid, and wherein the one or more amplicons respectively correspond to one or more target regions of the viral nucleic acid; (b) contacting the one or more amplicons with one or more probes under a condition conducive to a hybridization reaction to form one or more probe-amplicon hybrids; (c) determining a melting temperature (Tm) of each of the one or more probe-amplicon hybrids; (d) determining a difference between the melting temperature of each of the one or more probe-amplicon hybrids and a reference melting temperature corresponding to the same probe-amplicon hybrid; (e) determining an aggregated difference of melting temperatures of the one or more probe-amplicon hybrids based on the difference between the melting temperature of each of the one or more probe-amplicon hybrids and the reference melting temperature corresponding to the same probe-amplicon hybrid; and (f) identifying the variant of the virus in the sample as a candidate variant, if the aggregated difference of the melting temperatures of the one or more probe-amplicon hybrids is identical to a reference aggregated difference of the candidate variant or if a difference between the aggregated difference of the melting temperatures of the one or more probe-amplicon hybrids and the reference aggregated difference of the candidate variant is less than a threshold value.

Also within the scope of this disclosure is a system for identifying a variant of a virus, such as SARS-CoV-2. In some embodiments, the system may be configured to: (i) amplify a viral nucleic acid of a variant of a virus in a sample with one or more primer pairs to obtain one or more amplicons, wherein each of the one or more primer pairs comprises a forward primer and a reverse primer, wherein the one or more primer pairs each specific for a target region of the viral nucleic acid, and wherein the one or more amplicons respectively correspond to one or more target regions of the viral nucleic acid; (ii) contact the one or more amplicons with one or more probes under a condition conducive to a hybridization reaction to form one or more probe-amplicon hybrids; (iii) determine a melting temperature (Tm) of each of the one or more probe-amplicon hybrids; (iv) determine a difference between the melting temperature of each of the one or more probe-amplicon hybrids and a reference melting temperature corresponding to the same probe-amplicon hybrid; (v) determine an aggregated difference of melting temperatures of the one or more probe-amplicon hybrids based on the difference between the melting temperature of each of the one or more probe-amplicon hybrids and the reference melting temperature corresponding to the same probe-amplicon hybrid; and (vi) identify the variant of the virus in the sample as a candidate variant, if the aggregated difference of the melting temperatures of the one or more probe-amplicon hybrids is identical to a reference aggregated difference of the candidate variant or if a difference between the aggregated difference of the melting temperatures of the one or more probe-amplicon hybrids and the reference aggregated difference of the candidate variant is less than a threshold value.

In some embodiments, the variant may be a SARS-C V-2 variant. In some embodiments, the SARS-CoV-2 variant may be selected from B.1.1.7 (20I, Alpha), B.1.351 (20H, Beta), P.1 (20J, Gamma), 8.1.617.2 (21A, Delta), and B.1.1.529 (21K, Omicron, BA.2, BA.4, and BA.5).

The term “primer” refers to any nucleic acid that is capable of hybridizing at its 3′ end to a complementary nucleic acid molecule and that provides a free 3′ hydroxyl terminus, which can be extended by a nucleic acid polymerase. As used herein, amplification primers are a pair of nucleic acid molecules that can anneal to 5′ or 3′ regions of a gene (plus and minus strands, respectively, or vice-versa) and contain a short region in between. Under appropriate conditions and with appropriate reagents, such primers permit the amplification of a nucleic acid molecule having the nucleotide sequence flanked by the primers. For in situ methods, a cell or tissue sample can be prepared and immobilized on a support, such as a glass slide, and then contacted with a probe that can hybridize to RNA. Alternative methods for amplifying nucleic acids corresponding to expressed RNA samples include those described in, e.g., U.S. Pat. No. 7,897,750.

As used herein, the term “oligonucleotide” refers to a short polynucleotide, typically less than or equal to 300 nucleotides long (e.g., in the range of 5 and 150, preferably in the range of 10 to 100, more preferably in the range of 15 to 50 nucleotides in length). However, as used herein, the term is also intended to encompass longer or shorter polynucleotide chains. An “oligonucleotide” may hybridize to other polynucleotides, therefore serving as a probe for polynucleotide detection, or a primer for polynucleotide chain extension.

The term “probe,” as used herein, refers to an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches, which will interfere with hybridization between the target sequence and the single-stranded nucleic acids described herein. However, if the number of mutations is so great that no hybridization can occur under even the least stringent of hybridization conditions, the sequence is not a complementary target sequence. A probe may be single-stranded or partially single and partially double-stranded. The strandedness of the probe is dictated by the structure, composition, and properties of the target sequence. Probes may be directly labeled or indirectly labeled, such as with biotin to which a streptavidin complex may later bind.

The term “detection probe” refers to an oligonucleotide having a sequence sufficiently complementary to its target sequence to form a probe:target hybrid (e.g., probe: amplicon hybrid) stable for detection under stringent hybridization conditions. A probe is typically a synthetic oligomer that may include bases complementary to a sequence outside of the targeted region, which do not prevent hybridization under stringent hybridization conditions to the target nucleic acid. A sequence non-complementary to the target may be a homopolymer tract (e.g., poly-A or poly-T), promoter sequence, restriction endonuclease recognition sequence, or sequence to confer desired secondary or tertiary structure (e.g., a catalytic site or hairpin structure), or a tag region which may facilitate detection and/or amplification. “Stable” or “stable for detection” means that the temperature of a reaction mixture is at least 2° C. below the melting temperature (Tm) of a nucleic acid duplex contained in the mixture, more preferably at least 5° C. below the Tm, and even more preferably at least 10° C. below the Tm.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules.

“Substantially complementary” means that a nucleic acid or oligonucleotide has a sequence containing at least 10 contiguous bases that are at least 80% (e.g., 85%, 90%, 95%, 96%, 97%, 98%, 99%, and 100%) to at least 10 contiguous bases in a target nucleic acid sequence so that the nucleic acid or oligonucleotide can hybridize or anneal to the target nucleic acid sequence under, e.g., the annealing condition of a PCR assay or probe-target hybridization condition. Complementarity between sequences may be expressed a number of base mismatches in each set of at least 10 contiguous bases being compared. The term “substantially identical” means that a first nucleic acid is at least 80% (e.g., 85%, 90%, 95%, 96%, 97%, 98%, 99%, and 100%) complementary to a second nucleic acid so that the first nucleic acid is substantially complementary to and is capable of hybridizing to the complement of the second nucleic acid under PCR annealing or probe-target hybridization conditions.

“Hybridization” or “hybridizing” or “hybridize” or “anneal” refers to the ability of completely or partially complementary nucleic acid strands to come together under specified hybridization conditions in a parallel or preferably antiparallel orientation to form a stable double-stranded structure or region (sometimes called a “hybrid” or “duplex” or “stem”) in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

In some embodiments, the step of amplifying may be performed by a polymerase chain reaction (PCR). In some embodiments, the step of amplifying may be performed by an asymmetric PCR.

As used herein, the term “asymmetric PCR” refers to the preferential PCR amplification of one strand of a DNA target by adjusting the molar concentration of the primers in a primer pair so that they are unequal. An asymmetric PCR assay produces a predominantly single stranded product and a smaller quantity of a double stranded product as a result of the unequal primer concentrations. As asymmetric PCR proceeds, the lower concentration primer is quantitatively incorporated into a double stranded DNA amplicon, but the higher concentration primer continues to prime DNA synthesis, resulting in continued accumulation of a single stranded product. Asymmetric PCR also includes the use of a single primer for amplification.

As used herein, the term “amplification” and its variants include any process for producing multiple copies or complements of at least some portion of a polynucleotide, the polynucleotide typically being referred to as a “template.” The template polynucleotide can be single stranded or double stranded. A template may be a purified or isolated nucleic acid, or may be non-purified or non-isolated. Amplification of a given template can result in the generation of a population of polynucleotide amplification products, collectively referred to as an “amplicon.” The polynucleotides of the amplicon can be single stranded or double stranded, or a mixture of both. Typically, the template will include a target sequence, and the resulting amplicon will include polynucleotides having a sequence that is either substantially identical or substantially complementary to the target sequence. In some embodiments, the polynucleotides of a particular amplicon are substantially identical, or substantially complementary, to each other; alternatively, in some embodiments, the polynucleotides within a given amplicon can have nucleotide sequences that vary from each other. Amplification can proceed in a linear or exponential fashion, and can involve repeated and consecutive replications of a given template to form two or more amplification products. Some typical amplification reactions involve successive and repeated cycles of template-based nucleic acid synthesis, resulting in the formation of a plurality of daughter polynucleotides containing at least some portion of the nucleotide sequence of the template and sharing at least some degree of nucleotide sequence identity (or complementarity) with the template. In some embodiments, each instance of nucleic acid synthesis, which can be referred to as a “cycle” of amplification, includes creating free 3′ end (e.g., by nicking one strand of a dsDNA), thereby generating a primer and primer extension steps; optionally, an additional denaturation step can also be included wherein the template is partially or completely denatured. In some embodiments, one round of amplification includes a given number of repetitions of a single cycle of amplification. For example, a round of amplification can include 5, 10, 15, 20, 25, 30, 35, 40, 50, or more repetitions of a particular cycle. In one exemplary embodiment, amplification includes any reaction wherein a particular polynucleotide template is subjected to two consecutive cycles of nucleic acid synthesis. The synthesis can include template-dependent nucleic acid synthesis.

Amplification may also include isothermal amplification. The term “isothermal” means conducting a reaction at a substantially constant temperature, i.e., without varying the reaction temperature in which a nucleic acid polymerization reaction occurs. Isothermal temperatures for isothermal amplification reactions depend on the strand-displacing nucleic acid polymerase used in the reactions. Generally, the isothermal temperatures are below the melting temperature (Tm; the temperature at which half of the potentially double-stranded molecules in a mixture are in a single-stranded, denatured state) of the predominant reaction product, i.e., generally 90° C. or below, usually between about 20° C. and 75° C., and preferably between about 30° C. and 60° C., or more preferably at about 37° C.

As used herein, the term “contacting” and its variants, when used in reference to any set of components, includes any process whereby the components to be contacted are mixed into same mixture (for example, are added into the same compartment or solution), and does not necessarily require actual physical contact between the recited components. The recited components can be contacted in any order or any combination (or sub-combination), and can include situations where one or some of the recited components are subsequently removed from the mixture, optionally, prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as a polymerase, a primer set or a probe, includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

In some embodiments, the sample may be obtained from a subject infected with a virus or suspected of being infected with a virus. In some embodiments, the sample may include a viral genomic RNA or fragment thereof, and wherein prior to the step of amplifying, the method may include a reverse transcription step by a reverse transcription-polymerase chain reaction (RT-PCR). In some embodiments, the method may include extracting the viral genomic RNA or fragment thereof from the sample.

As used herein, the term “subject” refers to any organism having a genome, such as a living animal, e.g., a mammal, which has been the object of diagnosis, treatment, observation or experiment. Examples of a subject can be a human, a livestock animal (beef and dairy cattle, sheep, poultry, swine, etc.), or a companion animal (dogs, cats, horses, etc).

As used herein, a “sample” refers to any biological fluid or tissue obtained from an organism (e.g., patient) or from components (e.g., blood) of an organism. The sample may be of any biological tissue, cell(s) or fluid. The sample may be a “clinical sample,” which is a sample derived from a subject, such as a human patient or veterinary subject. Useful biological samples include, without limitation, whole blood, saliva, urine, synovial fluid, bone marrow, cerebrospinal fluid, vaginal mucus, cervical mucus, nasal secretions, sputum, semen, amniotic fluid, bronchoalveolar lavage fluid, and other cellular exudates from a patient or subject. Such samples may further be diluted with saline, buffer or a physiologically acceptable diluent. Alternatively, such samples are concentrated by conventional means. Biological samples may also include sections of tissues, such as frozen sections taken for histological purposes. A biological sample may also be referred to as a “patient sample.” A biological sample may also include a substantially purified or isolated protein, membrane preparation, or cell culture.

As used herein, the term “reference” value (e.g., reference melting temperature) refers to a value that statistically correlates to a particular outcome when compared to an assay result. In some embodiments, the reference value can be determined from statistical analysis that examines the mean of wild type values. The reference value may be a threshold score value or a cutoff score value. Typically a reference value will be a threshold above (or below) which one outcome is more probable and below which an alternative outcome is more probable.

In some embodiments, a difference of a value or level (e.g., melting temperature) may be a statistically significant difference between the quantities of an analyte present in a sample as compared to a control. For example, a difference may be statistically significant if the measured level of the analyte falls outside of about 1.0, 2.0, 3.0, 4.0, or 5.0 standard deviations of the mean of any control or reference group.

As used herein, the term “threshold value” refers to a point at which an analysis process may change and/or a point at which an action may be triggered. In some embodiments, the threshold value for the aggregated difference of melting temperature is between 1° C. and 10° C. (e.g., 1, 2, 3, 4, 5, 6, 7, 8, 9, or 10° C.).

In some embodiments, the step of amplifying, the reverse transcription step, and the step of contacting may be performed in a single reaction mixture. For example, the reverse transcription step and the amplification step may be performed using a QIAGEN One-Step RT-PCR kit (Qiagen cat. No 210212, Hilden, Germany).

In some embodiments, the step of amplifying for each of one or more primer pairs may be performed in separate reaction mixtures. For example, separate reaction mixtures may be prepared for each primer pair, such that detection of individual mutations in the viral nucleic acid is performed separately.

In some embodiments, at least one of one or more target regions may include one or more mutations characteristic of the variant of the virus. In some embodiments, one or more target regions may include a codon of L452 or a substitution thereof, a codon of E484 or a substitution thereof, a codon of N501 or a substitution thereof, or a combination thereof. In some embodiments, one or more target regions may include a codon of L452 or a substitution of L452Q car L452R, a codon of E484 or a substitution of E484K or E484A, a codon of N501 or a substitution of N501Y, or a combination thereof.

As used herein, a “target region,” “target nucleic acid sequence,” or “target sequence” refers to a specific sequence that may include all or part of the sequence of a single-stranded nucleic acid. A target sequence may be within a nucleic acid template or within the genome of a cell, which may be any form of single-stranded or double-stranded nucleic acid. A template may be a purified or isolated nucleic acid, or may be non-purified or non-isolated.

In some embodiments, one or more primer pairs may include a primer comprising nucleotide sequences selected from SEQ ID NOs: 1-2, 5-8, and 11-12, or comprising a nucleotide sequence having at least 80% (e.g., 80%, 82%, 84%. 86%, 88%, 90%, 91%, 92%, 93%, 94%, 95%, 96%, 97%, 98%, 99%) sequence identity with a nucleotide sequence selected from SEQ ID NOs: 1-2, 5-8, and 11-12.

In some embodiments, one or more primer pairs may include a first primer pair capable of hybridizing to a first target region of the viral nucleic acid. In some embodiments, one or more primer pairs may include a second primer pair capable of hybridizing to a second target region of the viral nucleic acid. In some embodiments, one or more primer pairs may include a third primer pair capable of hybridizing to a third target region of the viral nucleic acid.

In some embodiments, the first primer pairs may include respective nucleotide sequences of SEQ ID NOs: 1-2, or may include the respective nucleotide sequences having at least 80% (e.g., 80%, 82%, 84%, 86%, 88%, 90%, 91%, 92%, 93%, 94%, 95%, 96%, 97%, 98%, 99%) sequence identity with the nucleotide sequences of SEQ ID NOs: 1-2.

In some embodiments, the second primer pairs may include respective nucleotide sequences of SEQ ID NOs: 5-6 or SEQ ID NOs: 7-8; or may include the respective nucleotide sequences having at least 80% (e.g., 80%, 82%, 84%, 86%, 88%, 90%, 91%, 92%, 93%, 94%, 95%, 96%, 97%, 98%, 99%) sequence identity with the nucleotide sequences of SEQ ID NOs: 5-6 or SEQ ID NOs: 7-8.

In some embodiments, the third primer pairs may include respective nucleotide sequences of SEQ ID NOs: 11-12, or may include the respective nucleotide sequences having at least 80% (e.g., 80%, 82%, 84%, 86%, 88%, 90%, 91%, 92%, 93%, 94%, 95%, 96%, 97%, 98%, 99%) sequence identity with the nucleotide sequences of SEQ ID NOs: 11-12.

In some embodiments, the first target region of the viral nucleic acid may include a codon of L452 or a substitution of L452Q or L452R. In some embodiments, the second target region of the viral nucleic acid may include a codon of E484 or a substitution of E484K or E484A. In some embodiments, the third target region of the viral nucleic acid may include a codon of N501 or a substitution of N501Y.

In some embodiments, one or more amplicons may include a first amplicon comprising the first target region. In some embodiments, one or more amplicons may include a second amplicon comprising the second target region. In some embodiments, one or more amplicons may include a third amplicon comprising the third target region.

In some embodiments, one or more probes may include a first probe or a second probe capable of hybridizing to the first amplicon. In some embodiments, one or more probes may include a third probe or a fourth probe capable of hybridizing to the second amplicon. In some embodiments, one or more probes may include a fifth probe or a sixth probe capable of hybridizing to the third amplicon.

A probe can be made in various detection formats, such as dual labeled probes, including liner probes, Taqman probes, molecular beacon probes, and sloppy molecular beacon (SMB) probes. A “sloppy” probe refers to a probe that is mismatch-tolerant. Mismatch-tolerant probes hybridize with and generate a detectable signal for more than one target sequence at a detection temperature in an assay, and various hybrids so formed will have different melting points. Linear, or random coil, single-stranded probes are generally mismatch tolerant. Examples of such probes are hairpin or linear probes with an internal fluorescent moiety whose level of fluorescence increases upon hybridization to one or another target strand. See, e.g., U.S. Pat. Nos. 7,662,550 and 5,925,517. US 20130095479.

In some embodiments, the sloppy probes are dual-labeled hairpin probes or molecular beacon probes, described in U.S. Pat. Nos. 7,662,550 and 5,925,517. These hairpin probes contain a target binding sequence flanked by a pair of arms complementary to one another. They can be DNA, RNA, or PNA, or a combination of all three nucleic acids. Furthermore, they can contain modified nucleotides and modified internucleotide linkages. They can have a first fluorophore on one arm and a second fluorophore on the other arm, wherein the absorption spectrum of the second fluorophore substantially overlaps the emission spectrum of the first fluorophore. Such hairpin probes may be “molecular beacon probes” that have a fluorophore on one arm and a quencher on the other arm such that the probes are dark when free in solution. They can also be wavelength-shifting molecular beacon probes with, for example, multiple fluorophores on one arm that interact by fluorescence resonance energy transfer (FRET), and a quencher on the other arm. The target binding sequences can be, for example, 12 to 50, or 25 to 50 nucleotides in length, and the hybridizing arms can be 4 to 10 or 4 to 6 (e.g., 5 or 6) nucleotides in length. Molecular beacon probes can be tethered to primers, as described in U.S. Pat. Nos. 7,662,550 and 5,925,517 and WO 01/31062.

Sloppy molecular beacon probes thus refer to such a class of fluorescently labeled hairpin oligonucleotide hybridization probes. Such probes produce a detectable signal in a homogeneous assay, that is, without having to separate probes hybridized to target from unbound probes. By virtue of their ability to bind to more than one variants of a given target sequence, the probes can be used in assays to detect the presence of one variant of a nucleic acid sequence segment of interest from among a number of possible variants or even to detect the presence of two or more variants. The probes can therefore be used in combinations of two or more in the same assay. Because they differ in target binding sequence, their relative avidities for different variants are different. For example, a first probe may bind strongly to a wild-type sequence, moderately to a first allele, weakly to a second allele and not at all to a third allele; while a second probe may bind weakly to the wild-type sequence and the first variant, and moderately to the second variant and the third variant. Additional sloppy probes will exhibit yet different binding patterns due to their different target binding sequences. Thus, fluorescence emission spectra from combinations of sloppy probes define different microbial strains or species, as well as allelic variants/mutation of genes.

As the sloppy probes reproducibly fluoresce with variable intensities after binding to different DNA sequences, combinations can be used in, for example, rapid, and sensitive nucleic acid amplification reaction assays (e.g., PCR-based assays) that identify multiple pathogens or variants in a single reaction container. It is understood, however, that the assays can also be performed on samples suspected of containing directly detectable amounts of unamplified target nucleic acids. This identification assay is based on analyzing the spectra of a set of partially hybridizing sloppy signaling probes, such as sloppy molecular beacon probes, each labeled with a fluorophore that emits light with a different wavelength optimum, to generate “signature spectra” of species-specific or variant-specific DNA sequences.

Using the probes, multiplexing can be achieved, for example, by designing a different allele-discriminating molecular beacon probe for each target and labeling each probe differentially. (See, e.g., U.S. Pat. Nos. 7,662,550 and 5,925,517, WO 01/31062, and Tyagi et al. (2000) Nature Biotechnology 18: 1191-1196). Mixtures of allele-discriminating probes, each comprising aliquots of multiple colors, extend the number of probe signatures. To that end, every molecular beacon-target hybrid with a unique melting temperature will have corresponding unique signal intensity at a defined temperature and concentration of probe and amplicon. Thus, a limited number of sloppy probes could be used as probes to identify many different possible target sequences in a real-time PCR assay. The probes can be added to the amplification reaction mixture before, during, or after the amplification. See U.S. Pat. No. 7,662,550.

In some embodiments, the probes may include one or more labels. As used herein, a “label” or “reporter molecule” is a chemical or biochemical moiety useful for labeling a nucleic acid (including a single nucleotide), polynucleotide, oligonucleotide, or protein ligand, e.g., amino acid or antibody. Examples include fluorescent agents, chemiluminescent agents, chromogenic agents, quenching agents, radionucleotides, enzymes, substrates, cofactors, inhibitors, magnetic particles, and other moieties known in the art. Labels or reporter molecules are capable of generating a measurable signal and may be covalently or noncovalently joined to an oligonucleotide or nucleotide (e.g., a non-natural nucleotide) or ligand.

In some embodiments, the labels may include a fluorophore and/or a quencher. In some embodiments, the fluorophore is selected from fluorescein, cyanine 3, cyanine 5, TexasRed, and TAMRA. In some embodiments, the quencher is selected from BHQ1, BHQ2, and DABCYL.

In some embodiments, one or more probes may include a probe comprising nucleotide sequences selected from SEQ ID NOs: 3-4, 9-10, and 13-14, or comprising a nucleotide sequence having at least 80% (e.g., 80%, 82%, 84%, 86%, 88%, 90%, 91%, 92%, 93%, 94%, 95%, 96%, 97%, 98%, 99%) sequence identity with a nucleotide sequence selected from SEQ ID NOs: 3-4, 9-10, and 13-14.

In some embodiments, the first probe may include the nucleotide sequence of SEQ ID NO: 3, or may include a nucleotide sequence having at least 80% (e.g., 80%, 82%, 84%, 86%, 88%, 90%, 91%, 92%, 93%, 94%, 95%, 96%, 97%, 98%, 99%) sequence identity with the nucleotide sequence of SEQ ID NO: 3.

In some embodiments, the second probe may include the nucleotide sequence of SEQ ID NO: 4, or may include a nucleotide sequence having at least 80% (e.g., 80%, 82%, 84%, 86%, 88%, 90%, 91%, 92%, 93%, 94%, 95%, 96%, 97%, 98%, 99%) sequence identity with the nucleotide sequence of SEQ ID NO: 4.

In some embodiments, the third probe may include the nucleotide sequence of SEQ ID NO: 9, or may include a nucleotide sequence having at least 80% (e.g., 80%, 82%, 84%, 86%, 88%, 90%, 91%, 92%, 93%, 94%, 95%, 96%, 97%, 98%, 99%) sequence identity with the nucleotide sequence of SEQ ID NO: 9.

In some embodiments, the fourth probe may include the nucleotide sequence of SEQ ID NO: 10, or may include a nucleotide sequence having at least 80% (e.g., 80%, 82%, 84%, 86%, 88%, 90%, 91%, 92%, 93%, 94%, 95%, 96%, 97%, 98%, 99%) sequence identity with the nucleotide sequence of SEQ ID NO: 10.

In some embodiments, the fifth probe may include the nucleotide sequence of SEQ ID NO: 13, or may include a nucleotide sequence having at least 80% (e.g., 80%, 82%, 84%, 86%, 88%, 90%, 91%, 92%, 93%, 94%, 95%, 96%, 97%, 98%, 99%) sequence identity with the nucleotide sequence of SEQ ID NO: 13.

In some embodiments, the sixth probe may include the nucleotide sequence of SEQ ID NO: 14, or may include a nucleotide sequence having at least 80% (e.g., 80%, 82%, 84%, 86%, 88%, 90%, 91%, 92%, 93%, 94%, 95%, 96%, 97%, 98%, 99%) sequence identity with the nucleotide sequence of SEQ ID NO: 14.

In some embodiments, the aggregated difference of the melting temperature is determined based on an equation set forth as follows:
aggregated difference=\sqrt{Σᵢ₌₀ⁿ(Tmᵢ−Tmᵢ^{ref})²}, wherein Tmᵢ is a melting temperature determined for probe i, Tmᵢ^{ref} is a reference melting temperature determined for probe i, and n is an integer that denotes the number of probes.

In another aspect, this disclosure also provides an isolated nucleic acid, such as a primer or a probe, for example, for identifying a SARS-CoV-2 variant. In some embodiments, the isolated nucleic acid may include a nucleotide sequence of SEQ ID NO: 1-14 or may include a nucleotide sequence having at least 80% (e.g., 80%, 82%, 84%, 86%, 88%, 90%, 91%, 92%, 93%, 94%, 95%, 96%, 97%, 98%, 99%) sequence identity with a nucleotide sequence of SEQ ID NO: 1-14.

In some embodiments, the isolated nucleic acid may include a nucleotide sequence of SEQ ID NO: 5, 6, 9, 10, 13, and 14, or may include a nucleotide sequence having at least 80% (e.g., 80%, 82%, 84%, 86%, 88%, 90%, 91%, 92%, 93%, 94%, 95%, 96%, 97%, 98%, 99%) sequence identity with a nucleotide sequence of SEQ ID NO: 5, 6, 9, 10, 13, and 14.

In yet another aspect, this disclosure provides a kit comprising the isolated nucleic acid as disclosed herein. In some embodiments, the kit may include reagents for performing the above-described methods, including PCR and/or probe-target (e.g., probe-amplicon) hybridization reactions. To that end, one or more of the reaction components, e.g., PCR primers, polymerase, and probes, for the methods disclosed herein can be supplied in the form of a kit for use. In such a kit, an appropriate amount of one or more reaction components is provided in one or more containers or held on a substrate.

The kit also contains additional materials for practicing the above-described methods. In some embodiments, the kit contains some or all of the reagents and materials for performing a method that uses primers and/or probes according to this disclosure. Some or all of the components of the kits can be provided in containers separate from the container(s) containing the primers and/or probes of this disclosure. Examples of additional components of the kits include, but are not limited to, one or more different polymerases, one or more control reagents (e.g., probes or PCR primers or control templates), and buffers for the reactions (in 1× or concentrated forms). The kit may also include one or more of the following components: supports, terminating, modifying or digestion reagents, osmolytes, and an apparatus for detection.

The reaction components used can be provided in a variety of forms. For example, the components (e.g., enzymes, probes and/or primers) can be suspended in an aqueous solution or as a freeze-dried or lyophilized powder, pellet, or bead. In the latter case, the components, when reconstituted, form a complete mixture of components for use in an assay. The kits can be provided at any suitable temperature. For example, for storage of kits containing protein components (e.g., an enzyme) in a liquid, it is preferred that they are provided and maintained below 0° C., preferably at or below −20° C., or otherwise in a frozen state.

A kit or system of this disclosure may contain, in an amount sufficient for at least one assay, any combination of the components described herein. In some applications, one or more reaction components may be provided in pre-measured single-use amounts in individual, typically disposable, tubes or equivalent containers. With such an arrangement, a PCR assay can be performed by adding a target nucleic acid or a sample/cell containing the target nucleic acid to the individual tubes directly. The amount of a component supplied in the kit can be any appropriate amount, and may depend on the target market to which the product is directed. The container(s) in which the components are supplied can be any conventional container that is capable of holding the supplied form, for instance, microfuge tubes, ampoules, bottles, or integral testing devices, such as fluidic devices, cartridges, lateral flow, or other similar devices.

The kits can also include packaging materials for holding the container or a combination of containers. Typical packaging materials tor such kits and systems include solid matrices (e.g., glass, plastic, paper, foil, micro-particles, and the like) that hold the reaction components or detection probes in any of a variety of configurations (e.g., in a vial, microliter plate well, microarray, and the like). The kits may further include instructions recorded in a tangible form for use of the components.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

“Nucleic acid” or “oligonucleotide” or “polynucleotide” as used herein refers to at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single-stranded or double-stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine, and isoguanine. Nucleic acids may be obtained by chemical synthesis methods or by recombinant methods.

A “nucleic acid duplex,” “duplex,” “stem,” “nucleic acid hybrid,” or “hybrid” refers to a stable nucleic acid structure comprising a double-stranded, hydrogen-bonded region, e.g., RNA:RNA, RNA:DNA, and DNA:DNA duplex molecules and analogs thereof. Such structure may be detected by any known means, e.g., by using a labeled probe, an optically active probe-coated substrate sensitive to changes in mass at its surface (U.S. Pat. No. 6,060,237), or binding agents (U.S. Pat. No. 5,994,056).

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity is assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

The terms “determining,” “measuring,” “assessing,” and “assaying” are used interchangeably and include both quantitative and qualitative measurement, and include determining if a characteristic, trait, or feature is present or not. Assessing may be relative or absolute. “Assessing the presence of” a target includes determining the amount of the target present, as well as determining whether it is present or absent.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same is embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of this disclosure.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the disclosure. The upper and to lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

Ethical considerations. The usage of de-identified clinical samples from RT-PCR confirmed COVID-19 positive and negative patients in this study was approved by the Rutgers University institutional Review Board (IRB) under protocol numbers 20170001218 and 2020001541.

Viral cultures, RNA, and synthetic targets. SARS-CoV-2 RNA or viral cultures (USA WA1/2020 (WT, NR52285), B.1.1.7 (Alpha, NR54000), B.1.351 (Beta, NR-55282), P.1(Gamma, NR-54982), B.1.617.2 (Delta, NR-55611), and B.1.529 (Omicron BA.1, NR-56461), and B.1.529 (Omicron BA.2, NR-56520) were obtained from BEI Resources, NIAID (Manassas, VA).

RNA was isolated from the variant strains in a BSL3 laboratory using RNAdvance viral RNA extraction kit (Beckman Coulter, Indianapolis, IN). The extracted RNA was quantified using digital PCR using the primers specific for N1 gene.

For initial assay design and optimization, 50 by target sequences corresponding to a portion of the spike gene encoding either the 484K or 484A allele were synthesized (Millipore Sigma, The Woodlands, TX). As a test surrogate for SARS-CoV-2 Omicron RNA, an 89 bp single-stranded DNA sequence containing the 484-codon was also synthesized based on the reference sequence EPI_ISL_6842157 (GISAID).

Assay design, primers, and probes. SMBs and primers verified to detect mutations in codon N501 and E484 were used. An additional assay to detect the L452R (T22917G) mutation present in B.1.617.2 (Delta) was designed similarly as described previously (Banada P, et al. 2021. Journal of Clinical Microbiology 59:e00845-21.). Briefly, a total of 412,389 high quality SARS-CoV-2 genome sequences, deposited in GISAID (Elbe S, et at. 2017. Global challenges (Hoboken, NJ) 1:33-46.) as of Feb. 19, 2021, were analyzed using BLAST (Altschul S F, et al. 1997. Nucleic Acids Res 25:3389-402) and aligned with MAFFT (Katoh K, et al. 2002. Nucleic Acids Res 30:3059-66). Primers and probes were designed on the basis of sequence conservation using the Primer3 program (Untergasser A, et al. 2012. Nucleic acids research 40:e115-e115) to amplify a 122 by region flanking the position 22917 (codon 452) in the reference strain (GenBank accession number MN-908947). SMB probe design was performed using the web servers DNA MFOLD (http://www.unafold.org/mfold/applications/dna-folding-form.php) and DINAmelt (http://www. unafold.org/hybrid2.php) to predict the probe folding structures and probe-target hybrid Tm values, respectively.

A similar genome analysis was performed for the B.1.529 (Omicron) strain, comparing it to other variant strains at the primer/probe binding regions. As of Dec. 14, 2021, GISAID had 4486 B.1.529.1 (Omicron BA.1) and 9 BA.2 genomes. Unfortunately, most genomes contained repeat is NNNN's across the regions flanking the target codons, making the analysis difficult. Excluding those ‘dirty’ sequences, 4048 were analyzed for 484 mutations and 3,964 genomes for 501. Of these ‘clean’ sequences, 3985 (98.4%) had E484A, and 3946 (99.5%) had N501Y mutations. 100% of the 9 genomes in BA.2 carried both N501 Y and E484A SNPs. All analyzed genomes for 452 were wild-type.

In silica two-state melting hybridizations were performed to understand the Tm variations using the DINAmelt application. The final list of primers and probes used in this study are listed in Table 1. All assays were run as separate reactions. Primers were obtained from Millipore Sigma, and SMBs were synthesized by LGC Biosearch technologies (Petaluma, CA). An internal control (IC) assay developed by CDC, targeting the human RNase P gene was simultaneously performed for each extracted RNA specimen as a separate reaction in a separate well, using the TaqMan real-time PCR assay probe tagged with FAM at the 5′ end and Dabcyl quencher at the 3′ end.

SMB-assay formulation. TaqPath™ 1-Step RT-qPCR Master Mix, CG (ThermoFisher Scientific, Waltham, MA) was supplemented with the assay primers and probes at final concentrations, as mentioned in Table 1, for an asymmetric one-step RT PCR. A 1 μl of the template RNA was added per 20 μl reaction. The internal control contained primers and a probe specific for human RNase P as described previously (Emery S L, et al. 2004. Emerg Infect Dis 10:311-316; Wylie A L, et al. 2020. N Engl J Med 383:1283-1286.).

Analytical sensitivity. Each reaction was run in replicates of 4 in 384-well plates in a Roche LightCycler 480 (Roche, Indianapolis, IN). The one-step RT-PCR amplification was performed with the same PCR conditions described previously (Banada P, et al. 2021. Journal of Clinical Microbiology 59:e00845-21) and mentioned in Table 1. The total assay time was 1 h and 17 min. Automated Tm calls were made by each of the instrument Tm detection software at the completion of the assay, which were matched with the pre-established Tm-signature for the wild-type or the mutant variants. The pre-quantitated genomic RNA from the SARS-CoV-2 USA WA1/2020 (WT) and B.1.617.2 (Delta) and B.1.529 (Omicron) were diluted in Tris-EDTA (TE) buffer. For the background matrix, total nucleic acids were extracted from a SARS-CoV-2 negative nasopharyngeal (NP) specimen (confirmed negative by Xpert Xpress SARS-CoV-2 test). Reference RNA from Delta/Omicron BA.1/Omicron BA.2 were spiked to the negative matrix. Delta RNA was spiked at final concentrations of 200, 100, 20, 2 and 0.2 GE/μl and Omicron RNA (BA.1/BA.2) was spiked at final concentration ranging from 10³ to 1 GE/μl. Each dilution was tested in replicates of 8. A 1 μl aliquot of this mix was added to 19 μl of the one-step RT-PCR mix containing the primers and probes and was evaluated in the SMB-501/SMB-484/ SMB-452 assays. The LOD was defined at 95% positive rate on the non-linear regression fit curve analysis.

Clinical specimen evaluation and RT-PCR instrument feasibility. A total of 46 banked clinical samples were tested from October 2020 through April 2021 (Table 3a), and an additional 90 banked specimens were tested after April 2021 through September 2022 (Table 3b). These samples contained deidentified nasopharyngeal (NP) swabs, nasal swabs, and saliva obtained from patients undergoing routine COVID-19 clinical testing at the CLIA and CAP certified laboratories at the Public Health Research Institute (PHRI) and University Hospital, Newark, NJ, were selected for this study. RT-PCR cycle threshold (Ct) values at collection, ranged from a minimum Ct of 12.4 through a maximum Ct of 37.6 and were collected from the months of April 2021 through September 2022. All specimens were tested in Roche LightCycler 480 (LC480, Roche, Indianapolis, IN). Additionally, Thirty-four of these specimens from were used for testing in various RT-PCR instruments with all 3 assays. The RT-PCR instruments used were a Bio-Rad CFX96 (Bio-Rad, Hercules, California), Applied Biosystems™ 7500 (Thermo Fisher Scientific), and a Rotor Gene Q (Qiagen, Germantown, MD) located in PHRI laboratories, NJMS genomic laboratory and the UH molecular diagnostics laboratory. These instruments were selected based on the availability and accessibility for this testing. RNA was extracted from all the specimens using a QIAamp viral RNA isolation kit (Qiagen) or a QIASymphony DSP viral RNA extraction kit in a QIAsymphony automated instrument (Qiagen) according to the manufacturers recommendations, and a 5 μl volume of this extracted RNA was added to the one-step RT-PCR mix containing the primers and probes (Wyllie A L, et al. 2020. N Engl J Med 383:1283-1286). Each specimen was run with all 3 assays (SMB-501, SMB-484, and SMB-452) in separate wells. All instruments were programmed with the similar protocol as LC480 as mentioned in the Table 1. The internal control targeting RNaseP was run for all specimens. A reference Tm code was established for each SMB assay on all platforms using the WT genomic RNA, Alpha, Beta, Delta, and Omicron BA.1 RNA. Specimens that tested positive for SARS-CoV-2 wildtype or a VOC were confirmed by Sanger sequencing at the Department of Genomic Medicine, Rutgers Biomedical and Health Sciences, Newark using the primer pair: F-5′aggctgcgttatagcttgga3′ (SEQ ID NO: 15) and R-5′aaacagttgctggtgcatgt3′ (SEQ ID NO: 16) which amplifies a 284bp segment of the S-gene inclusive of the amino acid positions at 452, 484, and 501. Sequencing data were analyzed using Ugene (ver 37) or MegAlign Pro software (DNAStar, ver16). Seven representative clinical specimens (VSAP75, VSAP76, VSAP79, VSAP80, VSAP84, VSAP85, and VSAP90) were submitted for whole genome sequencing to the Department of Genomic Medicine, Rutgers Biomedical and Health Sciences, Newark to confirm the identification of Omicron subvariants, and a Delta subvariant sample. The sample library prep was prepared using the Qiagen QIAseq Direct SARS-CoV-2 kit (Qiagen, Cat #333891, Germantown, MD). Random-primed cDNA synthesis was performed on the viral RNA, followed by high-fidelity multiplex PCR. The 250bp enriched amplicon pools were amplified and indexed with unique dual indices. The sequencing was run on the illumina Miniseq nextGeneration sequencer using a 300-cycle kit and analyzed using the SARS-CoV-2 workflow in the QIAGEN CLC Genomics Workbench program. The FASTA files were used to create a phylogenic tree in Nextclade CLI 2.5.0, Nextclade Web 2.5.0.

Statistical analysis. Standard statistical analyses (average, standard deviation) and graphing were performed using Microsoft Excel and GraphPad Prism 8.4.3 for Windows, R version 4.1.1, and the ggplot2 package.

MACRO for identifying VOC based on Tm values. For identification and classification of the VOCs, a Microsoft Excel-based program was developed. Briefly, the program finds the closest match between the Tm signature from the patient specimens to that of the reference VOCs. A distance index (D-value) is calculated based on the difference in values between the reference and the unknown. A D-value of <5 was considered in these studies as a perfect match and ≥5 was classified as “indeterminate.” The program uses the Ct value of the internal control (IC) to assess failed run and a successful run. An “invalid” call is made if the IC fails to generate a Ct along with the SMB probes, and a “SARS-CoV-2 Not Detected” call is made if the N1 gene fails to generate a Ct. For VOC classification, the Tm signature values that are generated from each of the six SMB probes (two SMB for each of the three codons) are entered, and the tool generates an output result of either ‘Variant Indeterminate’/‘Wild type (Ancestral)’/‘B.1.1.7 (Alpha)’/‘B.1.351 (Beta)’/‘B.1.617.2 (Delta)’/ or ‘B.1.529 (Omicron BA.1)’/‘B.1.529 (Omicron BA.2)’/‘B.1.529 (Omicron BA.2.12.1)’/ or ‘B.1.529 (Omicron BA.4/5)’. A ‘Variant Indeterminate’ output is obtained if the Tm values are outside of the reference window (as described above) or if a Tm value of zero is entered for >2 SMB probes due to the failure of these probes to generate a Tm. However, if only 1 or 2 of the SMB probes fail to generate a Tm, the tool matches the remaining Tm values to the closest reference and reports the identified VOC as ‘presumptive.’

The Excel Analyze Tool utilizes an Excel Workbook (Tables 7A-D) that consists of two Worksheets (Tabs), one Tab, called “Data Input & Analysis,” is visible to the end-user, and one Tab, called “Calibrators,” is hidden (and not visible for the end-user). The Data Input & Analysis Tab allows the end-user to manually enter for each sample the Threshold Cycle (Ct) obtained for the Internal Control during the PCR step of the assay (Cell C10) and the Melting Temperatures (Tm's) obtained for each of the six molecular beacon probes during the denaturation profile analysis step of the assay (Cells E10, F10, G10, H10, I10, and J10) in Table A. In Cell D10, the Ct for the N1 gene (CoV-N probe) is entered. In case no Ct for the Internal Control or no Tm for one or more of the molecular beacon probes is obtained, the input fields for the Ct or any of the Tm's can be left empty, or “0”, or “ND” can be entered.

A formula (Cell D12) on the Data Input & Analysis Tab first determines if the assay is INVALID. An assay result (displayed in Cell D12) is determined to be “INVALID” if, for all six molecular beacon probes, no melting temperature (empty field, 0, or ND) is entered AND if no threshold value (empty field, 0, or ND) for the Internal Control is entered and the assay is determined to be INVALID if for all six molecular beacon probes, no melting temperature (empty field, 0, or ND) is entered AND if a threshold value for the Internal Control is entered that is 35.01 or higher.

An assay result (Cell D12) is determined to be “SARS-CoV-2 Negative” if, for all six molecular beacon probes, no melting temperature (empty field, 0, or ND) is entered AND if a threshold value for the Internal Control is entered that is between 0.01 and 35.

Pressing and releasing the “ANALYZE DATA” button on the Data input & Analysis Tab activates the MACRO “ANALYZE.”

The following paragraphs refer to content and actions on the Calibrators Tab.

The Tm values entered in Cells E10, F10, G10, H10, I10, and J10 on the Data Input & Analysis Tab are automatically transferred to Table B, Cells C11, D11, E11, F11, G11, and H11 on the (hidden) Calibrators Tab. The transferred value is automatically converted to “0” if the original cell on the Data Input & Analysis Tab is empty or “ND” is entered.

In Table C, on the Calibrator Tab, up to 200 calibrator species (wild-type, variants, or presumptive variants) can be entered (in Rows 19 to 218), together with the six melting temperatures of the six molecular beacon probes for the entered calibrator specie (Columns C to H). The data for the calibrator species can be entered by a user with administrative access to the hidden Calibrator tab.

In Table D (Rows 430 to 629), the Workbook automatically places the names of the calibrator species in Table C, Column B (Rows 19 to 218) in Column B (Cells B223 to B422). In Table D, the Workbook automatically calculates for each calibrator species listed in Table C (Rows 19 to 218) the square value of the difference in melting temperature of probe 1 (Table C, Column C) and the observed melting temperature of probe 1 for the sample (value in Table B, Cell C11) and place the resulting value in Column C of the same row. It repeats the same square value calculations of the difference in melting temperatures of probes 2 to 6 (Table C, Column D to H) and the observed melting temperatures of probes 2 to 6 for the sample (values in Table B, Cells D11, E11, F11, G11, and H11, respectively) and place the resulting values in Column D to H of the same row. In Table D, Column I, for each calibrator species, it calculates the square root of the sum of the values in Column C to H. This value is the “D-value.” The closer the sample relates to a listed calibrator species, the lower the D-value. A perfect match, in which all six molecular beacon probes show the same melting temperature as the six molecular beacon probes for a calibrator species, will return a D-value of 0. The higher the D-value, the more distant the strain found in the sample is from the calibrator species.

In Table E (Rows 430 to 629), the Workbook automatically places the values from Table D, Column I (Cells I223 to I422) in Column B (Cells B430 to B629), and the names of the calibrator species in Table D, Column B (Cells B223 to B422) in Column C (Cells C430 to C629).

Table F (Rows 634 to 833) is updated when the MACRO is activated on the Data Input & Analysis Tab. The MACRO (listed below) selects and copies Cells B430 to C629 (in Table F), and paste the copied values (utilizing Excel's Paste Special function) of Cells B430 to C629 in Cells B634 to C833 (in Table F). The MACRO then selects Cells B633 to C833 (in Table F) and utilizes Excel's Sort function to rearrange Column B (D-value) and Column C (calibrator species) in Table F from low to high D-values (ascending order).

As a result of the MACRO execution, Cell B634 will display the lowest D-value, and Cell C634 will display the corresponding calibrator species. The MACRO will reset the cursor to Cell C11 and Cell C13 will display the name of the calibrator species (updated by the MACRO in C634), and Cell E13 will display the D-value (updated by the MACRO in B634).

If the sum of the values of Cells C11, D11, E11, F11, G11, and H11 equals 0, then Cell C13 will display “SARS-CoV-2 Negative.” If the D-value in Cell E13 is 5 or higher, Cell C13 will not display any calibrator species (the cell value be empty).

The following paragraphs refer back to content and actions on the Data Input & Analysis Tab.

The second function of the activated MACRO is to re-align and re-center the Ct and Tm values entered by the end-user in Cells C10 to J10. Cell D12 will display the content of Cell C13 in the Calibrators Tab (the Workbook determined calibrator species). The content will only be displayed if the Workbook, as described above, does not determine that the assay is INVALID. Cell F12 will display “Indeterminate” if three, four, or five molecular beacon probes did not return a melting temperature OR if the D-value in Cell E13 in the Calibrators Tab is 5 or higher.

The codes for the MACRO are described below:

```
Sub ANALYZE( )
‘
‘ANALYZE Macro
‘
′ Keyboard Shortcut: Ctrl+Alt+s
‘
  Application.ScreenUpdating = False
    Sheets(“Calibrators”).Visible = True
    Sheets(“Calibrators”).Select
    Range(“b430:c629”).Select
  Selection.Copy
    Sheets(“Calibrators”).Select
    Range(“b634”).Select
  Selection.PasteSpecial Paste:=xlValues, Operation:=xlNone,
    SkipBlanks:= _ False, Transpose:=False
  Sheets(“Calibrators”).Select
  Range(“b633:c833”).Select
  Application.CutCopyMode = False
  Selection.Sort Key1:=Range(“b633”), Order1:=xlAscending,
    Header:=xlGuess _ , OrderCustom:=1, MatchCase:=False,
    Orientation:=xlTopToBottom
  Sheets(“Calibrators”).Select
  Range(“c11”).Select
  Sheets(“Calibrators”).Visible = False
  Sheets9”Data Input & Analysis”).Select
  Range(“c10:j10”).Select
  With Selection
    .HorizontalAlignment = xlGeneral
    .VerticalAlignment = xlBottom
    .WrapText = False
    .Orientation = 0
    .AddIndent = False
    .IndentLevel = 0
    .ShrinkToFit = False
    .ReadingOrder = xlContext
    .MergeCells = False
  End With
  With Selection
    .HorizontalAlignment = xlGeneral
    .VerticalAlignment = xlCenter
    .WrapText = False
    .Orientation = 0
    .AddIndent = False
    .IndentLevel = 0
    .ShrinkToFit = False
    .ReadingOrder = xlContext
    .MergeCells = False
  End With
  With Selection
    .HorizontalAlignment = xlCenter
    .VerticalAlignment = xlCenter
    .WrapText = False
    .Orientation = 0
    .AddIndent = False
    .IndentLevel = 0
    .ShrinkToFit = False
    .ReadingOrder = xlContext
    .MergeCells = False
  End With
  With Selection
    .HorizontalAlignment = xlGeneral
    .VerticalAlignment = xlCenter
    .WrapText = False
    .Orientation = 0
    .AddIndent = False
    .IndentLevel = 0
    .ShrinkToFit = False
    .ReadingOrder = xlContext
    .MergeCells = False
  End With
  With Selection.Font
    .Name = “Arial”
    .Size = 11
    .Strikethrough = False
    .Superscript = False
    .Subscript = False
    .OutlineFont = False
    .Shadow = False
    .Underline = xlUnderlineStyleNone
    .ColorIndex = xlAutomatic
    .TintAndShade = 0
    .ThemeFont = xlThemeFontNone
  End With
  With Selection.Font
    .Name = “Arial”
    .Size = 14
    .Strikethrough = False
    .Superscript = False
    .Subscript = False
    .OutlineFont = False
    .Shadow = False
    .Underline = xlUnderlineStyleNone
    .ColorIndex = xlAutomatic
    .TintAndShade = 0
    .ThemeFont = x1ThemeFontNone
  End With
  With Selection
    .HorizontalAlignment = xlCenter
    .VerticalAlignment = xlCenter
    .WrapText = False
    .Orientation = 0
    .AddIndent = False
    .IndentLevel = 0
    .ShrinkToFit = False
    .ReadingOrder = xlContext
    .MergeCells = False
  End With
  With Selection.Interior
    .Pattern = xlNone
    .TintAndShade = 0
    .PatternTintAndShade = 0
  End With
    Range(“c10”).Select
      End Sub
```

### Example 2

Limit of detection. The limit of detection (LoD) for the codon 452 (SMB-452 assay) was established by spiking various log dilutions of Delia reference strain (NR-5561) RNA at concentrations ranging from 10⁵ through 0.1 ge/reaction in Roche LC480. Each dilution was tested in replicates of four. The data was analyzed and the LoD was established based on the Tm values and melt peak heights (MPH) from both WT and MT probes. The Tm values for the WT probe (Cy3, λ533-580) and MT probe in the presence of the Delta target was 58.3° C.±0.12 and 63.2° C.±0.13, respectively forming a mutant detection signature for 452 assay. The overall LoD tar the SMB-452 assay, defined as the lowest target concentration where all 4 replicates were positive was found to be 1 GE. The LoD for detecting mutations in codons 501 (SMB-501 assay) and 484 (SMB-484 assay) was tested for Omicron with Omicron RNA in log-fold dilutions ranging from concentrations of 10⁵−1 ge/reaction in the presence of COVID-19 negative matrix. Based on the criteria for LoD established the LoD with SMB-501 assay was 100 ge/reaction (and with SMB-484 assay was 10⁴ ge/reaction The LOD of the new SMB-452 assay was established with serial dilutions of the Delta reference strain B.1.617.2 (NR-55611) RNA in negative nasal swab matrix at concentrations ranging from 200 through 0.2 genome equivalents (GE)/reaction in a Roche LC480. Delta B1.617.2 is WT in both the SMB-501 and SMB-484 assays (Table 3b). The Tm values for the new 452-WT probe (Cy3, λ533-580) and new 452-MT probe (Cy5, λ618-660) in the presence of the Delta target were 58.3° C.±0.12 and 63.2° C.±0.13, respectively defining a mutant to detection signature for SMB-452 assay. Based on the fit curves, the overall LOD combining all 3 mutation assays was found to be 20 GE. Similarly, an analytical LOT) was established for the SMB-VOC assay with Omicron BA.1 and BA.2 RNA at concentrations of 10³ through 1 GE/reaction in the presence of COVID-19 negative nasal swab matrix. A combined assay LOD of 22 GE and 36 GE/reaction for Omicron BA.1 and Omicron BA.2, respectively was established respectively based on the LOD criteria as described above.

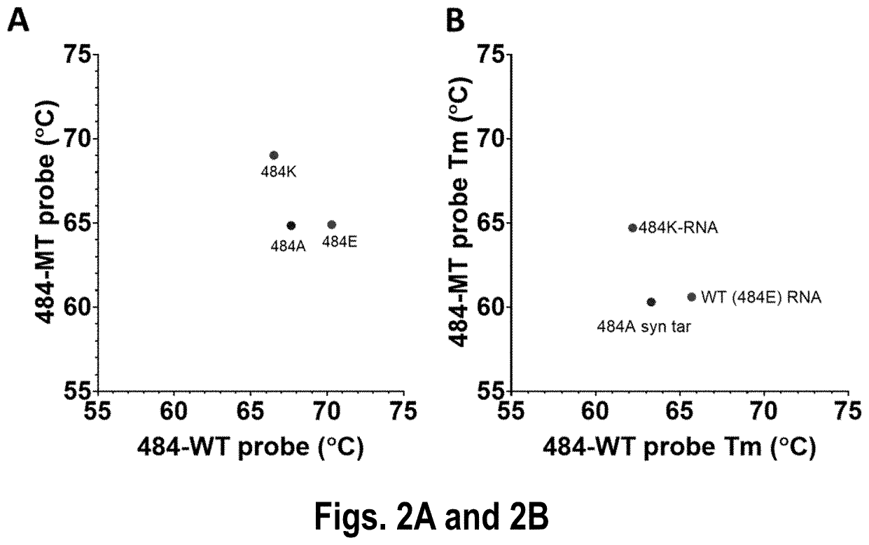

Evaluation of the SMB 484 assay for other variants. The SMB 484 assay was evaluated with synthetic targets and RNA encoding the codon 484 alleles 484K (Beta and Gamma), 484A (Omicron), along with the 484E WT sequence. First, a probe-target melt analysis (FIG. 2A) was performed in an LC480 instrument. A clear shift was observed in the Tm code for the Omicron 484A allele (67.6° C. WT-probe and 64.8° C. MT-probe) compared to the Beta and Gamma 484K allele (66.5° C. WT-probe and 69° C. MT-probe) and the WT 484E allele (WT-probe and 64.9° C. MT probe 70.2° C.). Thus, the Tm (ΔTm) difference between 484A and 484K (WT) was 1.1° C. with the WT probe and 4.4° C. with the MT probe, and between 484A and 484E was ˜2.5° C. with WT probe. These results were the same whether the assays were tested with RNA or synthetic targets (FIG. 2B). The unique Tm code for each mutation demonstrates that the assay, can specifically detect and differentiate between Omicron and other variants.

Tm code definition. The values produced by all SMBs against the reference WT and the MT SARS-CoV-2 strains generated in different instruments are listed in Tables 3a-b. The mean and standard deviations shown were derived from at least 4 replicates. Tm values can vary slightly between the samples and different instrument platforms. The 2- or 3-probe Tm coding approach provides for a robust sequence identification even in the presence of these Tm fluctuations, as shown in Tables 3a-b. For example, in the LC480 instrument, both WT and Delta have the same WT 501 allele, which results in a mean Tm of 59.7 for the 501-WI probe and 58.9 with the 501-MUT probe, but Alpha and Beta have a mutant 501N allele which results in a mean Tm of 55.7 for the 501-WI probe and 62.6±0.08° C. with the 501-MUT probe. Finally, Omicron has a mutant 501 allele which results in a mean Tm of 48.7° C.±0.16 for the 501-WT probe and 56.3° C.±0.18 with the 501-MUT probe. Similarly, Tm codes are established for other codons and both WT and variant alleles, as also shown in Tables 3a and 3b. In the testing procedure, samples that produced a Tm which was not within ±2° C. of an established reference Tm were either repeated or defined as an indeterminant sample.

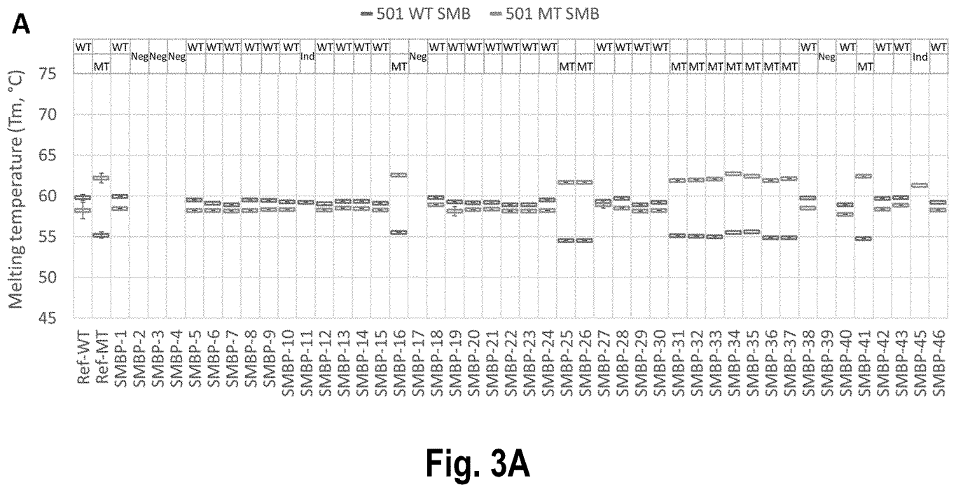

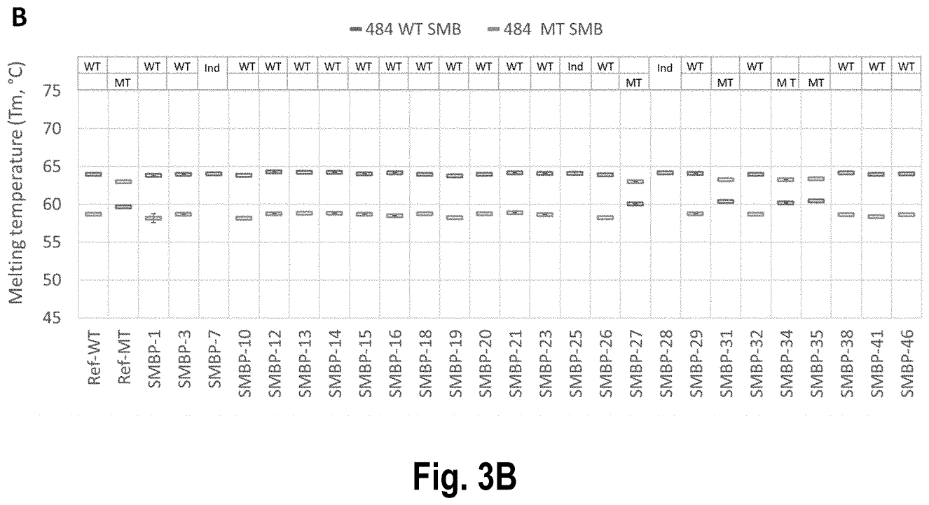

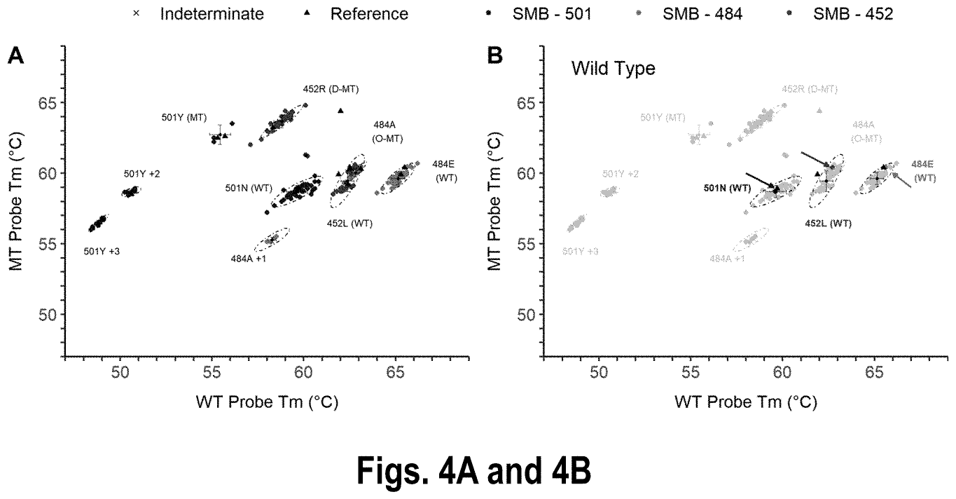

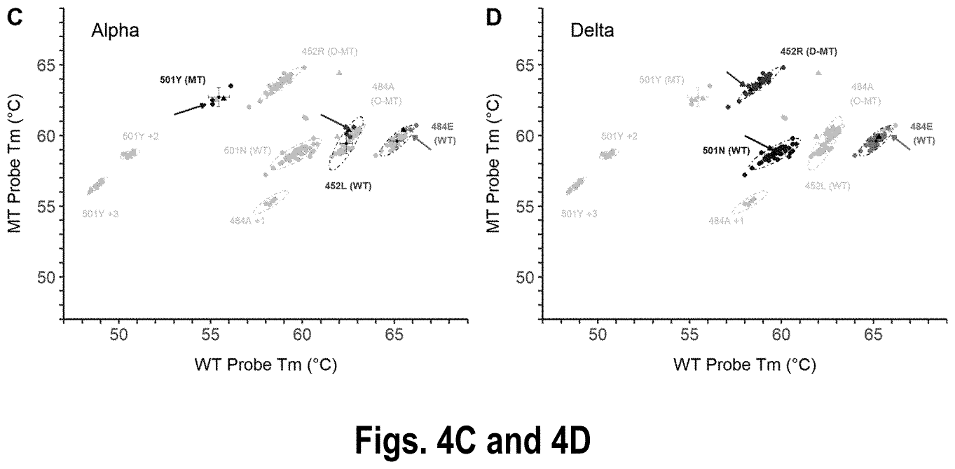

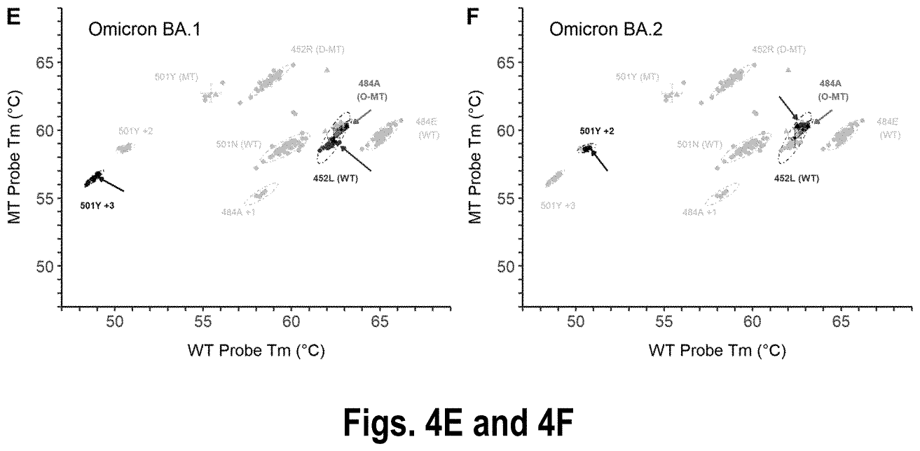

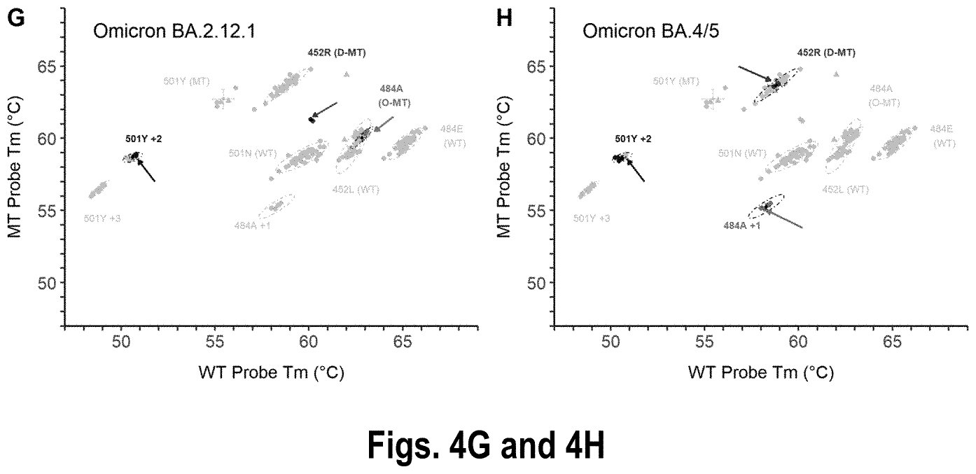

Validation with patient samples. As shown in Tables 3a and 3b, a total of 136 confirmed COVID-19 positive patient specimens and 39 confirmed CoV-2 negative specimens were tested in a Roche LightCycler 480 to evaluate the clinical performance of the SMB-VOC assay. The sample source, collection timeline, and the initial Ct value at collection were also recorded (Table 3a and b). 46 specimens collected from October 2020-March 2021 and 90 specimens from April 2021-September 2022 were tested, with Ct values ranging from 12 to 38.3. All VOCs were identified as described earlier, using the Excel analyze tool to transform Tm values into a variant identification. The Tm values obtained from all 3 component assays, and identification for each patient sample tested are listed in Tables 3a-b. The clustering of various mutations and the establishment of a Tm signature to identify specific WT or VOCs using the disclosed assay (SMB-VOC assay) was demonstrated in FIG. 3 and FIG. 4. Sanger sequencing of all specimens (Tables 3a and Table 5) was used to confirm the target codon sequence (either WI or mutant) in each specimen. Of the 46 specimens tested (between October 2020-March 2021), there was a 100% concordance of the mutation detection with the Sanger sequencing. Of the 90 samples tested since April 2021, compared to Sanger Sequencing for the target mutations (based on the signature in Table 2), the LC480 correctly identified 51/56 (91%) of the Delta variants as Delta, correctly identified all 16/16 (100%) Omicron BA.1 variants as Omicron BA.1, all 6/6 (100%) Omicron BA.2 variants as BA.2, all 3/3 (100%) Omicron BA.2.12.1 variants as Omicron BA.2.12.1, and all 3/3 (100%) Omicron BA.4/5 variants as Omicron BA.4/5. The SMB-VOC assay results call was set with high stringency, so any specimen failing to yield a Tm in >2 probes or producing a Tm that falls outside of a defined Tm window will result in an indeterminate call. The five specimens (VSAP36, VSAP38, VSAP39, VSAP43, VSAP79) that produced indeterminate results by SMB-VOC assay (Table 4) were identified as Delta by Sanger/whole genome sequencing (Table 5). None of the specimens were incorrectly identified as WT or incorrectly identified as a different VOC. Thus, excluding specimens with indeterminant results, the SMB-VOC assay was 100% sensitive and 100% specific. Including the five indeterminate specimens, the SMB-VOC assay sensitivity was reduced to 94.3%, without changing the specificity (Table 6).

To further understand the adaptability of the assay in diagnostic and hospital laboratories, we further validated the method in three additional commonly used RT-PCR instruments with melt capability. The performance of these new test instruments was compared to the LC480 as the gold standard. A total of 34 out of the 90 COVID-19 positive patient specimens were tested with all three defined assays (SMB-501, SMB-484, and SMB-452) on different RT-PCR instruments (Table 4). The BioRadCFX96 identified 97% (33/34) of the specimens correctly, and one sample tested negative (Kappa, k=0.9). The ABI7500 identified 32/34 (94%) of the specimens correctly (k=0.9), one sample was negative, and the other was indeterminate. However, the Qiagen rotor-geneQ (RGQ) instrument performed relatively poorly, where it identified 67.6% (23/34) of the specimens correctly (k=0.39) and 11/34 (32.4%) of the specimens were indeterminates. Thus, the SMB-VOC assay is highly adaptable and reproducible in most RT-PCR instruments with melt capability.

Discussion

Although whole-genome sequencing is a powerful tool to identify new viral lineages, an efficient, high-throughput screening test that accurately identifies VOC provides many advantages. This example demonstrates a efficient and easily adaptable assay capable of detecting the rapidly emerging Omicron variants, in addition to Alpha, Beta, and Gamma strains. The disclosed assay is sensitive, specific, and has high throughput. It can be performed on most qRT-PCR instruments once reference Tm values are established, unlike almost all commercial assays (Vogels C B F, et al. 2021. PLoS Biol 19:e3001236; Neopane P, et al. 2021. Infect Drug Resist 14:4471-4479). It was hypothesized that mutations at the codons 501 and 484 would be common in other emergent SARS-CoV-2 variants, and mutations at both codons are repeated in Omicron. Although Delta predominantly remained wild type at these codons, mutations at codon 452 similar to the CAL.20C variant observed first in California are considered a key Delta-defining mutation. The mutations on these codons are shown to be responsible for the increased infectivity, transmission, escape humoral immunity, and reduced susceptibility to monoclonal antibody treatments, and data is not clear yet on their resistance to antivirals. The disclosed assay is capable of detecting mutations in these codons and will help with surveillance to track the variants and may also help guide targeted therapy.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 1
Primers, probe, and PCR conditions.

| | | | | | | | Final | Thermal |
|---|---|---|---|---|---|---|---|---|
| | | | | | | | conc. | cycling |
| | | SEQ | | | | Ampli- | per | and post |
| | Primer/ | ID | | | | con | reac- | PCR melt |
| Assay | Probe | NO | 5′ | Sequence | 3′ | size | tion | conditions |
| SMB- | 501-F | 1 | | Ggttttaattgttactttcctttacaa | | 89 bp | 0.2 μM | Uracil DNA glucosylase |
| 501 | 501-R | 2 | | Gaaagtactactactctgtatggttgg | | | 2 μM | incubation: 37° C.- |
| | 501-WT | 3 | Quasar | CCGCgtt[pdU]ccatcccactaatgc | BHQ-2 | | 0.4 μM | 2 min; |
| | | | 570 | tg[pdU]tggttaccaacGCGG | | | | Reverse transcription |
| | 501-MT | 4 | Quasar | CGCGgtt[pdu\|ccatcccacttatgc | BHQ-2 | | 0.4 μM | (RT): 50° C.-15 min; |
| | | | 670 | tg[pdu]tggttaccaacCGCG | | | | Asymmetric PCR: |
| | | | | | | | | Denaturation: 95° C.- |
| SMB- | 484-Fw* | 5 | | ctatcaggccggtaRcaMac | | 76 bp | 0.2 μM | 3 s; |
| 484 | 484-Rw* | 6 | | gaaacYatatgatYgtaaaggaaag | | | 5 μM | Annealing/extension: |
| | 484-F | 7 | | Ctatcaggccggtagcacac | | | | 58º C.-30 s. |
| | 484-R | 8 | | Gaaaccatatgattgtaaaggaaag | | | | Cycles: 45 Post- |
| | 484-WT | 9 | Quasar | CCGCGccttgtaatggtgttgaagg | BHQ-2 | | 0.4 μM | PCR melt: |
| | | | 570 | ttttaattgttacGCGCGG | | | | Denaturation: |
| | 484-MT | 10 | Quasar | CCGCGccttgtaatggtgttaaagg | BHQ-2 | | 0.4 μM | 95° C.-30 s; |
| | | | 670 | ttttaattgttacGCGCGG | | | | Melt: 45° C. to |
| | | | | | | | | 85º C.; |
| SMB- | 452-F | 11 | | Aggctgcgttatagcttgga | | 122 bp | 0.1 μM | Acquisitions: |
| 452 | 452-R | 12 | | cagttgaaatctctctctcaaaaggt | | | 2.5 μM | Continuous at 2/° C. |
| | 452-WT | 13 | Quasar | CCGCGtaattataattacctgtatag | BHQ-2 | | 0.4 μM | |
| | | | 570 | attgtttaggaagtcCGCGG | | | | |
| | 452-MT | 14 | Quasar | CGCCGAggtggtaattatataattac | BHQ-2 | | 0.4 μM | |
| | | | 670 | cggtatagattgtttaggTCGGCG | | | | |

The lowercase letter indicates the SMB probe region, the uppercase letter indicates the SMB stem region, and pdU -C5 Propynyl-deoxyuridine and BHQ indicate “black hole quencher.”
*wobble primers: R = A + G; M = A + C; Y = C + T.

TABLE 2
Differentiating mutations on the 3 signature codon probe
binding regions on the S-gene to specifically identify
and differentiate all SARS-CoV-2 variants of concern.

| | 501 | 484 | 452 |
|---|---|---|---|
| Alpha | N501Y | E484 (WT) | L452 (WT) |
| Beta | N501Y | E484K | L452 (WT) |
| Delta | N501, Q498S | | L452R |
| Omicron: | G496S, Q498R, | E484A | WT |
| BA.1 | N501Y, Y505H | | |
| Omicron: | Q498R, N501Y, | E484A | WT |
| BA.2 | Y505H | | |
| Omicron: | Q498R, N501Y, | E484A | L452Q |
| BA.2.12.1 | Y505H | | |
| Omicron: | Q498R, N501Y, | E484A, F486V | L452R |
| BA.4/5 | Y505H | | |

TABLE 3a
SMB-VOC Assay and Sanger sequencing results from COVID positive clinical samples
detecting the mutations at 501 and 484 codons (October 2020-April 2021).

| | | | SMB-501 assay | | | SMB-484 assay | | | Confirmation by sequencing1 | | |
|---|---|---|---|---|---|---|---|---|---|---|---|
| | | | WT probe | MT probe | | WT probe | MT probe | | | N501Y | E484K |
| | Date of | | (Cy3) | (Cy5) | | (Cy3) | (Cy5) | | | mutant | mutant |
| | 1st test | PCR | Tm | Tm | | Tm | Tm | | Wild | (AAT- | (GAA- |
| Samples | (dd/mm/yy) | Ct* | (° C.) | (° C.) | Id | (2° C.) | (° C.) | Id | type | TAT) | AAA) |
| WT-Reference | | | 59.8 ± 0.4 | 58.2 ± 1 | WT | 64 ± 0.1 | 58.7 ± 0.4 | WT | Yes | No | No |
| MT-Reference | | | 55.2 ± 0.4 | 62.2 ± 0.6 | MT | 59.7 ± 0.5 | 63 ± 0.6 | MT | No | Yes | No |
| SMBP-1 | October- | 29.1 | 59.9 | 58.4 | WT | 63.9 | 58.2 | WT | Yes | No | No |
| SMBP-2 | November | 34.8 | NP | NP | Neg | | | | | | |
| SMBP-3 | 2020 | 34.5 | NP | NP | Neg | 64.0 | 58.7 | WT | | | |
| SMBP-4 | | 35.4 | NP | NP | Neg | | | | | | |
| SMBP-5 | | 39.2 | 59.5 | 58.2 | WT | | | | | | |
| SMBP-6 | | 36.0 | 59.1 | 58.2 | WT | | | | Yes | No | No |
| SMBP-7 | | 33.7 | 58.9 | 58.1 | WT | 64.0 | | Ind | | | |
| SMBP-8 | | 36.8 | 59.5 | 58.2 | WT | | | | | | |
| SMBP-9 | | 27.9 | 59.4 | 58.3 | WT | | | | | | |
| SMBP-10 | January 2021 | 29.4 | 59.3 | 58.3 | WT | 63.8 | 58.2 | WT | Yes | No | No |
| SMBP-11 | | 39.1 | 59.9 | 58.6 | WT | | | | | | |
| SMBP-12 | | 33.8 | 59.0 | 58.2 | WT | 64.3 | 58.8 | WT | Yes | No | No |
| SMBP-13 | | 17.7 | 59.3 | 58.5 | WT | 64.2 | 58.9 | WT | Yes | No | No |
| SMBP-14 | | 21.3 | 59.4 | 58.5 | WT | 64.2 | 58.8 | WT | Yes | No | No |
| SMBP-15 | | 17.5 | 59.1 | 58.3 | WT | 64.0 | 58.7 | WT | Yes | No | No |
| SMBP-16 | | 30.0 | 55.5 | 62.6 | MT | 64.2 | 58.5 | WT | No | Yes | No |
| SMBP-17 | | 41.2 | ND | NP | Neg | | | | | | |
| SMBP-18 | | 23.2 | 59.8 | 58.9 | WT | 64.0 | 58.7 | WT | | | |
| SMBP-19 | | 28.3 | 59.3 | 58.1 | WT | 63.8 | 58.2 | WT | Yes | No | No |
| SMBP-20 | | 17.1 | 59.2 | 58.3 | WT | 64.0 | 58.8 | WT | Yes | No | No |
| SMBP-21 | | 16.4 | 59.2 | 58.4 | WT | 64.2 | 58.9 | WT | Yes | No | No |
| SMBP-22 | | 18.2 | 58.9 | 58.1 | WT | | | | Yes | No | No |
| SMBP-23 | | 21.1 | 58.9 | 58.1 | WT | 64.1 | 58.6 | WT | Yes | No | No |
| SMBP-24 | | 39.0 | 59.5 | 58.2 | WT | | | | | | |
| SMBP-25 | | 28.4 | 54.5 | 61.6 | MT | 64.1 | NP | Ind | No | Yes | No |
| SMBP-26 | February 2021 | 27.8 | 54.5 | 61.6 | MT | 63.9 | 58.3 | NP | No | Yes | No |
| SMBP-27 | | 33.5 | 59.3 | 58.9 | WT | 60.1 | 63.0 | MT | Yes | No | Yes |
| SMBP-28 | | 38.3 | 59.7 | 58.5 | WT | 64.1 | NP | Ind | ND | ND | ND |
| SMBP-29 | | 18.1 | 58.9 | 58.1 | WT | 64.1 | 58.8 | WT | Yes | No | No |
| SMBP-30 | | 27.8 | 59.2 | 58.2 | WT | | | | | | |
| SMBP-31 [symbol] | | 17.6 | 55.1 | 61.9 | MT | 60.4 | 63.3 | MT | No | Yes | Yes |
| SMBP-32 [symbol] | | 17.4 | 55.1 | 61.9 | MT | 64.0 | 58.7 | WT | No | Yes | No |
| SMBP-33 | | 32.8 | 55.0 | 62.1 | MT | | | | | | |
| SMBP-34 | | 25.2 | 55.5 | 62.7 | MT | 60.2 | 63.3 | MT | No | Yes | Yes |
| SMBP-35 | | 18.4 | 55.6 | 62.5 | MT | 60.5 | 63.4 | MT | No | Yes | Yes |
| SMBP-36 | | 18.8 | 54.9 | 61.9 | MT | | | | No | Yes | No |
| SMBP-37 | | 28.4 | 54.9 | 62.2 | MT | | | | | | |
| SMBP-38 | | 16.4 | 59.8 | 58.5 | WT | 64.1 | 58.6 | WT | | | |
| SMBP-39 | | 32.2 | NP | NP | Neg | | | | | | |
| SMBP-40 | | 32.3 | 58.9 | 57.8 | WT | | | | Yes | No | No |
| SMBP-41 | March | 32.6 | 55.4 | 62.6 | MT | 63.9 | 58.3 | WT | No | Yes | No |
| SMBP-42 | 2021 | 36.8 | 59.7 | 58.4 | WT | | | | | | |
| SMBP-43 | | 30.1 | 59.8 | 58.9 | WT | | | | Yes | No | No |
| SMBP-44 | | 33.3 | NP | NP | Inv | | | | | | |
| SMBP-45 | | 34.6 | NP | 61.3 | Ind | | | | | | |
| SMBP-46 | | 23.3 | 59.2 | 58.285 | WT | 64.0 | 58.6 | WT | Yes | No | No |

IC—internal control; WT—wildtype; MT—Mutant; NP—No Tm peak; Neg—Negative; ND—not Inv—Invalid; Ind—Indeterminate.
*Xpert Xpress SARS-CoV-2 or Xpert Xpress SARS-CoV-2/Flu/RSV tests
1Representative strains were sequenced.
^{a}sequencing was repeated twice to confirm the identified mutations. Blocked cells- Corresponding samples that are not tested by the SMB-484 assay or by sequencing due to insufficient clinical sample remaining. [symbol]
indicates data missing or illegible when filed

TABLE 3b
Validation of the SMB-VOC assay using patient specimens in Roche LC480
(targeting SARS-CoV-2 VOCs after April 2021 until September 2022).

| Reference | | | Initial | | | | | | | | |
|---|---|---|---|---|---|---|---|---|---|---|---|
| strains/ | | | sample | Internal | SMB-501 Assay | | SMB-484AKw Assay | | SMB-452 Assay | | Final id |
| Patient | | Date of | PCR | control | WT | MT | WT | MT | WT | MT | by SMB VOC |
| samples | Source | collection | Ct | Ct | (Cy3) | (Cy5) | (Cy3) | (Cy5) | (Cy3) | (Cy5) | assay |
| USA-WAI/ | BEI NR52285 | | | | 59.7 ± | 58.9 ± | 65.5 ± | 60.4 ± | 61.9 ± | 59.9 ± | Wild type (Ancestral) |
| 2020 (WT) | | | | | 0.10 | 0.07 | 0.04 | 0.05 | 0.08 | 0.07 | |
| B.1.1.7 | BEI NR54000 | | | | 55.7 ± | 62.6 ± | 65.5 ± | 60.4 ± | 62.5 ± | 60.4 ± | B.1.1.7 (Alpha) |
| (Alpha) | | | | | 0.04 | 0.08 | 0.12 | 0.05 | 0.04 | 0.03 | |
| B.1.351 | BEI NR55282 | | | | 55.3 ± | 62.5 ± | 62.0 ± | 64.4 ± | 62.5 ± | 60.3 ± | B.1.351 (Beta) |
| (Beta) | | | | | 0.06 | 0.01 | 0.03 | 0.04 | 0.04 | 0.04 | |
| B.1.617.2 | BEI NR55611 | | | | 59.6 ± | 58.7 ± | 65.3 ± | 59.9 ± | 58.3 ± | 63.25 ± | B.1.617.2 (Delta) |
| (Delta) | | | | | 0.05 | 0.05 | 0.04 | 0.08 | 0.12 | 0.04 | |
| B.1.529/ | BEI NR56461 | | | | 49.0 ± | 56.7 ± | 63.1 ± | 60.3 ± | 62.4 ± | 59.2 ± | B.1.529 (Omicron BA.1) |
| BA.1 | | | | | 0.06 | 0.04 | 0.04 | 0.04 | 0.03 | 0.04 | |
| (Omicron) | | | | | | | | | | | |
| B.1.529/ | BEI NR56520 | | | | 50.6 ± | 58.5 ± | 63.1 ± | 60.3 ± | 62.5 ± | 60.3 ± | B.1.529 (Omicron BA.2) |
| BA.2 | | | | | 0.10 | 0.06 | 0.03 | 0.03 | 0.02 | 0.05 | |
| (Omicron) | | | | | | | | | | | |
| VSAP1 | NP | April 2021 | 27.7 | 27.7 | 59.6 | 58.7 | 65.6 | 60.4 | 62.7 | 60.4 | Wild type (Ancestral) |
| VSAP2 | NP | | 30.1 | 29.3 | 55.1 | 62.2 | 65.6 | 60.4 | 62.4 | 60.1 | B.1.1.7 (Alpha) |
| VSAP3 | NP | May 2021 | 30.3 | 29.2 | 60.4 | 59.1 | 65.3 | 59.8 | 59.2 | 64.2 | B.1.617.2 (Delta) |
| VSAP4 | NP | | 32.0 | 25.6 | 55.1 | 62.5 | 65.9 | 60.4 | 62.6 | 59.9 | B.1.1.7 (Alpha) |
| VSAP5 | Saliva | | 25.7 | 23.7 | 60.4 | 58.5 | 65.8 | 60.5 | 58.5 | 63.5 | B.1.617.2 (Delta) |
| VSAP6 | Saliva | | 33.2 | 26.9 | 56.1 | 63.5 | 66.2 | 60.7 | 62.8 | 60.6 | B.1.1.7 (Alpha) |
| VSAP7 | NP | June 2021 | 20.0 | 29.6 | 60.6 | 59.8 | 65.3 | 60.0 | 60.1 | 64.8 | B.1.617.2 (Delta) |
| VSAP8 | NP | July 2021 | 27.2 | 29.7 | 60.0 | 59.1 | 65.2 | 59.3 | 59.2 | 64.0 | B.1.617.2 (Delta) |
| VSAP9 | NP | | 28.2 | 30.3 | 60.1 | 59.1 | 65.2 | 59.4 | 59.3 | 64.2 | B.1.617.2 (Delta) |
| VSAP11 | NP | | 19.5 | 30.1 | 59.6 | 58.9 | 65.6 | 60.0 | 58.9 | 63.8 | B.1.617.2 (Delta) |
| VSAP12 | NP | | 31.5 | 26.5 | 58.9 | 58.8 | 65.2 | 59.3 | 58.9 | 63.7 | B.1.617.2 (Delta) |
| VSAP13 | NP | | 23.3 | 30.2 | 59.9 | 59.0 | 65.2 | 59.8 | 59.3 | 64.0 | B.1.617.2 (Delta) |
| VSAP14 | NP | | 27.4 | 26.1 | 60.1 | 59.2 | 65.2 | 59.6 | 59.2 | 64.2 | B.1.617.2 (Delta) |
| VSAP15 | NP | | 28.0 | 30.3 | 60.2 | 58.9 | 65.0 | 59.2 | 59.0 | 64.0 | B.1.617.2 (Delta) |
| VSAP16 | NP | | 21.9 | 30.4 | 59.8 | 59.0 | 65.1 | 59.8 | 59.2 | 63.9 | B 1.617.2 (Delta) |
| VSAP17 | NP | | 37.6 | 33.9 | No Tm | | | | | | ND |
| VSAP18 | NP | | 23.3 | 28.6 | 59.7 | 59.0 | 65.3 | 59.8 | 59.1 | 63.9 | B.1.617.2 (Delta) |
| VSAP19 | Saliva | | 22.7 | 24.4 | 59.3 | 58.6 | 65.0 | 59.2 | 58.5 | 63.4 | B.1.617.2 (Delta) |
| VSAP20 | Saliva | August 2021 | 26.3 | 24.1 | 59.4 | 58.5 | 65.1 | 59.9 | 58.5 | 63.3 | B.1.617.2 (Delta) |
| VSAP21 | Saliva | | 15.1 | 25.5 | 58.0 | 57.2 | 65.5 | 59.8 | 57.1 | 62.0 | B.1.617.2 (Delta) |
| VSAP22 | Saliva | | 26.1 | 28.8 | 59.4 | 58.5 | 64.9 | 59.0 | 58.6 | 63.3 | B.1.617.2 (Delta) |
| VSAP23 | Saliva | | 18.5 | 23.5 | 58.8 | 58.0 | 65.1 | 59.7 | 58.1 | 62.9 | B.1.617.2 (Delta) |
| VSAP24 | Saliva | | 36.7 | 27.2 | 60.8 | 59.4 | 65.6 | No Tm | 59.0 | 64.4 | Pres B.1.617.2 (Delta) |
| VSAP25 | NP | | 18.0 | 30.9 | 59.8 | 58.9 | 65.4 | 59.7 | 59.1 | 63.9 | B.1.617.2 (Delta) |
| VSAP26 | NS | | 23.2 | 31.9 | 60.6 | 59.4 | 65.0 | 59.3 | 59.4 | 64.3 | B.1.617.2 (Delta) |
| VSAP27 | NP | September | 18.1 | 28.7 | 60.0 | 59.2 | 65.4 | 60.0 | 59.3 | 64.1 | B.1.617.2 (Delta) |
| VSAP28 | NS | 2021 | 21.2 | 30.1 | 59.7 | 58.8 | 65.2 | 60.0 | 58.9 | 63.9 | B.1.617.2 (Delta) |
| VSAP29 | Saliva | | 18.1 | 30.8 | 58.8 | 58.0 | 65.2 | 59.3 | 58.0 | 63.0 | B.1.617.2 (Delta) |
| VSAP30 | NP | | 17.3 | 27.1 | 59.8 | 58.9 | 65.6 | 59.9 | 59.2 | 64.0 | B.1 617.2 (Delta) |
| VSAP31 | NP | | 16.5 | 22.2 | 59.9 | 59.1 | 65.3 | 60.0 | 59.0 | 63.8 | B.1.617.2 (Deita) |
| VSAP32 | NP | | 21.2 | 29.7 | 59.6 | 58.9 | 65.6 | 60.1 | 59.1 | 63.9 | B.1.617.2 (Delta) |
| VSAP33 | NP | | 30.4 | 24.9 | 60.2 | 59.2 | 65.0 | 59.4 | 59.2 | 64.1 | B.1.617.2 (Delta) |
| VSAP34 | NP | October | 23.6 | 26.9 | 60.6 | 58.9 | 65.2 | 59.9 | 59.1 | 63.9 | B.1.617.2 (Delta) |
| | | 2021 | | | | | | | | | |
| VSAP35 | Saliva | November | 12.4 | 22.3 | 59.2 | 58.4 | 64.9 | 59.7 | 58.8 | 63.5 | B.1.617.2 (Delta) |
| VSAP36 | NP | 2021 | 28.2 | 28.9 | 59.7 | 59.0 | 65.3 | 59.8 | No Tm | No.Tmn | VAR IND |
| VSAP37 | Saliva | | 22.7 | 27.2 | 59.4 | 58.5 | 64.9 | 59.3 | 59.0 | 63.8 | B.1.617.2 (Delta) |
| VSAP38 | NS | | 30.0 | 34.4 | 60.0 | 58.8 | 65.0 | No Tm | No Tm | No Tm | VAR IND |
| VSAP39 | NP | | 29.9 | 32.0 | 59.8 | 58.3 | 65.1 | No Tm | No Tm | No Tm | VAR IND |
| VSAP40 | Saliva | | 25.2 | 24.7 | 59.6 | 58.4 | 64.8 | 58.9 | 58.9 | 63.9 | B.1.617.2 (Delta) |
| VSAP41 | NP | | 22.7 | 31.2 | 59.9 | 58.7 | 65.2 | 59.4 | 59.1 | 63.9 | B.1.617.2 (Delta) |
| VSAP42 | Saliva | | 18.2 | 23.2 | 59.1 | 58.3 | 64.8 | 59.3 | 58.9 | 63.8 | B.1.617.2 (Delta) |
| VSAP43 | NP | | 29.8 | 34.4 | 59.7 | 58.8 | 65.0 | 59.2 | No Tm | No Tm | VAR IND |
| VSAP44 | NP | | 29.2 | 32.2 | 59.6 | 58.8 | 65.1 | 59.2 | 59.0 | 64.0 | B.1.617.2 (Delta) |
| VSAP45 | NP | December | 28.4 | 32.0 | 60.0 | 58.7 | 64.8 | NP | 58.8 | 64.0 | Pres. B.1.617.2 (Delta) |
| VSAP46 | NP | 2021 | 17.0 | 29.9 | 59.8 | 58.8 | 65.1 | 59.9 | 59.3 | 63.8 | B.1.617.2 (Delta) |
| VSAP47 | NP | | 22.5 | 28.9 | 60.5 | 58.8 | 65.1 | 59.5 | 58.7 | 63.5 | B.1.617.2 (Delta) |
| VSAP48 | Saliva | | 26.0 | 25.0 | 59.3 | 58.2 | 65.0 | NP | 58.8 | 63.5 | Pres. B.1.617.2 (Delta) |
| VSAP49 | NP | | 27.2 | 28.6 | 59.7 | 58.9 | 65.0 | 59.1 | 59.1 | 63.9 | B.1.617.2 (Delta) |
| VSAP50 | NP | | 20.1 | 30.6 | 59.6 | 58.8 | 65.1 | 59.7 | 59.0 | 63.8 | B.1.617.2 (Delta) |
| VSAP51 | NP | | 24.1 | 29.9 | 59.2 | 58.4 | 64.8 | 59.3 | 58.5 | 63.3 | B.1.617.2 (Delta) |
| VSAP52 | NP | | 15.7 | 23.5 | 59.2 | 58.5 | 65.0 | 59.5 | 58.5 | 63.4 | B.1.617.2 (Delta) |
| VSAP53 | NP | | 27.1 | 30.1 | 59.3 | 58.5 | 64.9 | 59.3 | 58.2 | 63.5 | B.1.617.2 (Delta) |
| VSAP54 | NP | | 27.3 | 30.0 | 48.9 | 56.5 | 62.7 | 59.8 | 62.3 | 58.9 | B.1.529 (Omicron BA.1) |
| VSAP55 | NP | | 28.4 | 30.8 | 59.1 | 58.4 | 64.8 | 59.6 | 58.5 | 63.4 | B.1.617.2 (Delta) |
| VSAP56 | NP | | 25.5 | 31.0 | 49.0 | 56.7 | 62.7 | 60.0 | 62.3 | 59.3 | B.1.529 (Omicron BA.1) |
| VSAP57 | NP | | 23.8 | 31.3 | 58.9 | 58.1 | 64.3 | 58.9 | 58.1 | 62.9 | B.1.617.2 (Delta) |
| VSAP58 | NP | | 22.9 | 30.9 | 59.1 | 58.4 | 64.6 | 59.3 | 58.3 | 63.0 | B.1.617.2 (Delta) |
| VSAP59 | NP | | 24.2 | 29.9 | 48.9 | 56.5 | 62.8 | 59.9 | 62.3 | 59.0 | B.1.529 (Omicron BA.1) |
| VSAP60 | NP | | 20.4 | 27.6 | 59.3 | 58.6 | 64.8 | 59.7 | 58.6 | 63.4 | B.1.617.2 (Delta) |
| VSAP61 | NP | | 26.4 | 28.5 | 48.8 | 56.5 | 62.6 | 60.0 | 62.2 | 58.9 | B.1.529 (Omicron BA.1) |
| VSAP62 | NP | | 24.7 | 29.4 | 48.5 | 56.1 | 62.6 | 59.8 | 62.1 | 58.7 | B.1.529 (Omicron BA.1) |
| VSAP63 | NP | | 23.4 | 28.7 | 48.8 | 56.4 | 62.7 | 59.9 | 62.2 | 58.8 | B.1.529 (Omicron BA.1) |
| VSAP64 | NP | | 21.4 | 79.1 | 59.1 | 58.4 | 64.8 | 59.5 | 58.5 | 63.4 | B.1.617.2 (Delta) |
| VSAP65 | NP | | 24.0 | 28.0 | 48.4 | 56.0 | 62.2 | 59.3 | 61.6 | 58.5 | B.1.529 (Omicron BA.1) |
| VSAP66 | NP | | 21.9 | 32.5 | 59.5 | 58.7 | 64.9 | 59.7 | 58.6 | 63.5 | B.1.617.2 (Delta) |
| VSAP67 | NP | | 23.1 | 28.4 | 59.3 | 58.5 | 64.8 | 59.7 | 58.6 | 63.4 | B.1.617.2 (Delta) |
| VSAP68 | NP | | 21.9 | 29.9 | 48.8 | 56.5 | 62.7 | 60.0 | 62.0 | 58.9 | B.1.529 (Omicron BA.1) |
| VSAP69 | NP | | 26.2 | 31.9 | 48.8 | 56.3 | 62.6 | 59.8 | 62.0 | 58.9 | B.1.529 (Omicron BA.1) |
| VSAP70 | NP | | 21.3 | 28.8 | 48.8 | 56.4 | 62.6 | 59.8 | 62.0 | 58.7 | B.1.529 (Omicron BA.1) |
| VSAP71 | NP | | 26.8 | 32 8 | 48.5 | 56.2 | 62.5 | 59.6 | 61.8 | 58.7 | B.1.529 (Omicron BA.1) |
| VSAP72 | NP | | 19.4 | 27.8 | 48.7 | 56.4 | 62.7 | 60.0 | 62.1 | 59.0 | B.1.529 (Omicron BA.1) |
| VSAP73 | NP | | 18.0 | 30.3 | 58.4 | 57.7 | 64.0 | 58.6 | 57.7 | 62.4 | B.1.617.2 (Delta) |
| VSAP74 | NP | | 18.2 | 29.9 | 48.8 | 56.3 | 62.9 | 60.0 | 62.1 | 58.9 | B.1.529 (Omicron BA.1) |
| VSAP75 | NP | | NK | 34.9 | 50.2 | 58.7 | 58.5 | 55.5 | 58.9 | 63.8 | B.1.529 (Omicron BA.4/5) |
| VSAP76 | NP | January 2220 | 21.5 | 27.0 | 49.0 | 56.8 | 63.1 | 60.4 | 62.5 | 59.1 | B.1.529 (Omicron BA.1) |
| VSAP77 | Saliva | February | 25.9 | 28.5 | 49.1 | 56.7 | 62.9 | 60.1 | 62.6 | 59.0 | B.1.529 (Omicron BA.1) |
| VSAP78 | NP | 2022 | 19.3 | 28.1 | 49.1 | 56.8 | 63.1 | 60.2 | 62.7 | 59.1 | B.1.529 (Omicron BA.1) |
| VSAP79 | NP | March 2022 | 21.6 | 31.4 | 59.8 | 58.8 | 62.8 | 58.3.64.0 | 58.9 | 63.9 | VAR. IND |
| VSAP80 | NP | April 2022 | 26.2 | 26.5 | 50.7 | 58.7 | 62.9 | 60.0 | 62.9 | 60.3 | B.1.529 (Omicron BA.2) |
| VSAP81 | NP | | 20.4 | 28.1 | 50.8 | 58.7 | 62.7 | 59.8 | 62.7 | 60.4 | B.1.529 (Omicron BA.2) |
| VSAP82 | NP | | 20.9 | 29.9 | 50.7 | 58.7 | 63.0 | 60.3 | 62.7 | 60.4 | B.1.529 (Omicron BA.2) |
| VSAP83 | Saliva | | 29.6 | 27.0 | No Tm | 58.4 | 63.0 | 60.2 | 62.6 | 60.2 | Pres B.1.529 (Omicron BA.2) |
| VSAP84 | NP | May 2022 | 25.2 | 28.2 | 50.6 | 58.6 | 58.2 | 55.1 | 58.9 | 63.8 | B.1.529 (Omicron BA.4/5) |
| VSAP85 | NP | | 24.9 | 30.7 | 50.8 | 58.8 | 63.0 | 60.3 | 60.1 | 61.3 | B.1.529 (Omicron BA.2.12.1) |
| VSAP86 | NP | | 25.8 | 30.3 | 50.7 | 58.8 | 63.0 | 60.4 | 60.1 | 61.3 | B.1.529 (Omicron BA.2.12.1) |
| VSAP87 | NP | | 21.8 | 30.9 | 50 8 | 58.9 | 63.1 | 60.5 | 60.2 | 61.2 | B.1.529 (Omicron BA.2.12.1) |
| VSAP88 | NP | January | 17.7 | 28.8 | 50.5 | 58.6 | 63.1 | 60.4 | 62.8 | 60.4 | B.1.529 (Omicron BA.2) |
| VSAP89 | NP | 2022 | 20.8 | 26.5 | 50.6 | 58.6 | 63.1 | 60.5 | 62.9 | 60.4 | B.1.529 (Omicron BA.2) |
| VSAP90 | NP | July 2022 | NK | 27.9 | 50.4 | 58.7 | 58.4 | 55.4 | 58.9 | 63.8 | B.1.529 (Omicron BA.4/5) |
| VSAP91 | NP | September | NK | 27.2 | 50.4 | 58.4 | 58.0 | 55.2 | 58.7 | 63.8 | B.1.529 (Omicron BA.4/5) |
| | | 2022 | | | | | | | | | |

Cov-2 Negatives

| | | | | | | |
|---|---|---|---|---|---|---|
| N-VSAP1 | NP | January 2021 | Neg | 28.9 | No Tm | SARS-CoV-2 Not |
| | | | | | | Detected |
| N-VSAP2 | NP | February | Neg | 25.0 | No Tm | SARS-CoV-2 Not |
| | | 2021 | | | | Detected |
| N-VSAP3 | NP | | Neg | 24.4 | No Tm | ARS-CoV-2 Not |
| | | | | | | Detected |
| N-VSAP4 | NP | | Neg | 22.9 | No Tm | SARS-CoV-2 Not |
| | | | | | | Detected |
| N-VSAP5 | NP | | Neg | 23.7 | No Tm | SARS-CoV-2 Not |
| | | | | | | Detected |
| N-VSAP6 | NP | | Neg | 29.9 | No Tm | SARS-CoV-2 Not |
| | | | | | | Detected |
| N-VSAP7 | NP | | Neg | 29.3 | No Tm | SARS-CoV-2 Not |
| | | | | | | Detected |
| N-VSAP8 | NP | | Neg | 25.5 | No Tm | SARS-CoV-2 Not |
| | | | | | | Detected |
| N-VSAP9 | NP | | Neg | 25.2 | No Tm | SARS-CoV-2 Not |
| | | | | | | Detected |

Ct—Cycle threshold;
NP—Nasopharyngeal swab specimen;
NS—Nasal Swab specimen;
NK—not known;
Neg—Negative;
VAR IND—Variant indeterminate
Pres.: Presumptive (presumptive call is made when one or more probes in the assay fails to give a Tm)

TABLE 4
Patient specimen validation in four different RT-PCR instruments with melt curve analysis
using 3 SMB assays for detection and differentiation of SARS-Cov-2 variants of concern.

| | | | | LC480 | | | | | | | CFX96 | | | | | | |
|---|---|---|---|---|---|---|---|---|---|---|---|---|---|---|---|---|---|
| | | | | SMB-501 | | SMB-484 | | SMB-452 | | | SMB-501 | | SMB-484 | | SMB-452 | | |
| | | | | Assay | | Assay | | Assay | | | Assay | | Assay | | Assay | | |
| Ref./Pt. | | | IC | WT | MT | WT | MT | WT | MT | Final | WT | MT | WT | MT | WT | MT | Final |
| samples | DOC | Source | Ct | (Cy3) | (Cy5) | (Cy3) | (Cy5) | (Cy3) | (Cy5) | id | (Cy3) | (Cy5) | (Cy3) | (Cy5) | (Cy3) | (Cy5) | id |
| WT-Reference | | BEI | | 59.7 | 58.9 | 65.7 | 60.6 | 61.9 | 59.9 | z | 59.0 | 58.0 | 65.0 | 59.8 | 62.0 | 60.0 | |
| | | NR52285 | | | | | | | | | | | | | | | |
| MT-B.1.1.7 | | BEI | | 55.7 | 62.6 | 65.7 | 60.6 | 61.9 | 59.9 | | 54.5 | 62.0 | 65.0 | 59.8 | 62.0 | 60.0 | |
| (Alpha) | | NR54000 | | | | | | | | | | | | | | | |
| MT-B.1.351 | | BEI NR- | | 55.7 | 62.6 | 62.2 | 64.7 | 61.9 | 59.9 | | 54.5 | 62.0 | 61.0 | 64.0 | 62.0 | 60.0 | |
| (Beta) | | 55282 | | | | | | | | | | | | | | | |
| MT-B.1.617.2 | | BEI NR- | | 59.7 | 58.9 | 65.7 | 60.6 | 58.3 | 63.3 | | 59.0 | 58.0 | 65.0 | 59.8 | 58.0 | 64.0 | |
| (Delta) | | 55611 | | | | | | | | | | | | | | | |
| MT-B.1.529 | | BEI NR- | | 49.0 | 56.6 | 63.4 | 60.6 | 61.9 | 59.9 | | | | | | | | |
| (Omicron) | | 56461 | | | | | | | | | | | | | | | |
| VSAP1 | April | NP | 27.7 | 59.6 | 58.7 | 65.5 | 60.4 | 62.7 | 60.4 | WT | 59.0 | 58.0 | 65.0 | 60.0 | 62.0 | 59.0 | WT |
| | 2021 | | | | | | | | | | | | | | | | |
| VSAP2 | April | NP | 29.3 | 55.1 | 62.2 | 65.3 | 60.1 | 62.4 | 60.1 | Alpha | 54.0 | 62.0 | 65.0 | 59.0 | 62.0 | 0.0 | P- |
| | 2021 | | | | | | | | | | | | | | | | Alpha |
| VSAP3 | May | NP | 29.2 | 60.4 | 59.1 | 65.9 | 60.6 | 59.2 | 64.2 | Delta | 59.0 | 57.0 | 65.0 | 59.0 | 58.0 | 64.0 | Delta |
| | 2021 | | | | | | | | | | | | | | | | |
| VSAP4 | May | NP | 28.6 | 55.1 | 62.5 | 65.6 | 60.3 | 62.6 | 59.9 | Alpha | 54.0 | 61.0 | 65.0 | 59.0 | 62.0 | 0.0 | P- |
| | 2021 | | | | | | | | | | | | | | | | Alpha |
| VSAP5 | May | Saliva | 23.7 | 50.4 | 58.5 | 65.3 | 60.2 | 58.9 | 63.5 | Delta | 59.0 | 58.0 | 65.0 | 60.0 | 58.0 | 64.0 | Delta |
| | 2021 | | | | | | | | | | | | | | | | |
| VSAP6 | May | Saliva | 26.9 | 56.1 | 63.5 | 66.9 | 61.5 | 62.8 | 60.6 | Alpha | 55.0 | 62.0 | 65.0 | 60.0 | 63.0 | 59.0 | Alpha |
| | 2021 | | | | | | | | | | | | | | | | |
| VSAP7 | June | NP | 29.6 | 60.6 | 59.8 | 66.6 | 61.3 | 60.1 | 64.8 | Delta | 60.0 | 59.0 | 65.0 | 61.0 | 59.0 | 65.0 | Delta |
| | 2021 | | | | | | | | | | | | | | | | |
| VSAP8 | July | NP | 29.7 | 60.0 | 59.1 | 65.8 | 60.5 | 59.2 | 64.0 | Delta | 59.0 | 58.0 | 65.0 | 59.0 | 0 | 64.0 | P- |
| | 2021 | | | | | | | | | | | | | | | | Delta |
| VSAP9 | July | NP | 30.3 | 60.1 | 59.1 | 65.9 | 60.7 | 59.3 | 64.2 | Delta | 59.0 | 58.0 | 65.0 | 60.0 | 58.0 | 64.0 | Delta |
| | 2021 | | | | | | | | | | | | | | | | |
| VSAP10 | July | NP | 34.7 | 60.2 | 59.3 | 68.3 | 61.0 | 59.5 | 64.5 | Delta | 60.0 | 58.0 | 65.0 | 60.0 | 58.0 | 64.0 | Delta |
| | 2021 | | | | | | | | | | | | | | | | |
| VSAP11 | July | NP | 30.1 | 59.6 | 58.9 | 65.6 | 60.4 | 58.9 | 63.8 | Delta | 59.0 | 58.0 | 65.0 | 59.0 | 58.0 | 63.0 | Delta |
| | 2021 | | | | | | | | | | | | | | | | |
| VSAP12 | July | NP | 76.5 | 58.9 | 58.8 | 65.6 | 60.3 | 58.9 | 63.7 | Delta | 59.0 | 58.0 | 65.0 | 59.0 | 58.0 | 64.0 | Delta |
| | 2021 | | | | | | | | | | | | | | | | |
| VSAP13 | July | NP | 30.2 | 59.9 | 59.0 | 65.9 | 60.7 | 59.3 | 64.0 | Delta | 59.0 | 58.0 | 65.0 | 60.0 | 0 | 64.0 | P- |
| | 2021 | | | | | | | | | | | | | | | | Delta |
| VSAP14 | July | NP | 26.1 | 60.1 | 59.2 | 66.1 | 60.8 | 59.2 | 64.2 | Delta | 59.0 | 58.5 | 65.0 | 60.0 | 58.0 | 64.0 | Delta |
| | 2021 | | | | | | | | | | | | | | | | |
| VSAP15 | July | NP | 30.3 | 60.2 | 58.9 | 65.8 | 60.6 | 59.0 | 64.0 | Delta | 59.0 | 58.0 | 65.0 | 60.0 | 58.0 | 64.0 | Delta |
| | 2021 | | | | | | | | | | | | | | | | |
| VSAP16 | July | NP | 80.4 | 59.8 | 59.0 | 65.8 | 60.6 | 59.2 | 63.9 | Delta | 59.0 | 58.0 | 65.0 | 60.0 | 58.0 | 64.0 | Delta |
| | 2021 | | | | | | | | | | | | | | | | |
| VSAP17 | July | NP | 33.9 | 0 | 0 | 65.9 | 60.3 | 0 | 0 | Neg | 0 | 0 | 0 | 0 | 0 | 0 | Neg |
| | 22021 | | | | | | | | | | | | | | | | |
| VSAP18 | July | NP | 28.6 | 59.7 | 59.0 | 65.7 | 60.5 | 59.1 | 63.9 | Delta | 59.0 | 58.0 | 65.0 | 60.0 | 58.0 | 64.0 | Delta |
| | 2021 | | | | | | | | | | | | | | | | |
| VSAP19 | July | Saliva | ND | 59.3 | 58.6 | 65.2 | 60.1 | 58.5 | 63.4 | Delta | 59.0 | 58.0 | 65.0 | 59.0 | 58.0 | 64.0 | Delta |
| | 2021 | | | | | | | | | | | | | | | | |
| VSAP20 | August | Saliva | ND | 59.4 | 58.5 | 65.3 | 60.1 | 58.5 | 63.3 | Delta | 59.0 | 58.0 | 65.0 | 60.0 | 58.0 | 64.0 | Delta |
| | 2021 | | | | | | | | | | | | | | | | |
| VSAP21 | August | Saliva | ND | 58.0 | 57.2 | 64.0 | 58.8 | 57.1 | 62.0 | Delta | 59.0 | 59.0 | 65.0 | 60.0 | 58.0 | 64.0 | Delta |
| | 2021 | | | | | | | | | | | | | | | | |
| VSAP22 | August | Saliva | ND | 59.4 | 58.5 | 65.4 | 60.1 | 58.6 | 63.3 | Delta | 59.0 | 58.0 | 65.0 | 59.0 | 58.0 | 64.0 | Delta |
| | 2021 | | | | | | | | | | | | | | | | |
| VSAP23 | August | Saliva | ND | 58.8 | 58.0 | 64.8 | 59.7 | 58.1 | 62.9 | Delta | 60.0 | 59.0 | 66.0 | 60.0 | 58.0 | 64.0 | Delta |
| | 2021 | | | | | | | | | | | | | | | | |
| VSAP24 | August | Saliva | 27.2 | 60.8 | 59.4 | 66.3 | 60.9 | 59.0 | 64.4 | Delta | 0 | 0 | 65.0 | 0 | 0 | 0 | Neg |
| | 2021 | | | | | | | | | | | | | | | | |
| VSAP25 | August | NP | 30.9 | 59.8 | 58.9 | 65.8 | 60.8 | 59.1 | 63.9 | Delta | 59.0 | 58.0 | 65.0 | 60.0 | 58.0 | 64.0 | Delta |
| | 2021 | | | | | | | | | | | | | | | | |
| VSAP26 | August | Nasal | 31.3 | 60.6 | 59.4 | 66.1 | 61.0 | 59.4 | 64.3 | Delta | 59.0 | 57.0 | 65.0 | 0 | 0 | 64.0 | P- |
| | 2021 | | | | | | | | | | | | | | | | Delta |
| VSAP27 | September | NP | 28.7 | 60.0 | 59.2 | 66.0 | 60.7 | 59.3 | 64.1 | Delta | 59.0 | 58.0 | 65.0 | 60.0 | 58.0 | 64.0 | Delta |
| | 2021 | | | | | | | | | | | | | | | | |
| VSAP28 | September | Nasal | 30.3 | 59.7 | 58.8 | 65.7 | 60.6 | 58.9 | 63.9 | Delta | 59.0 | 58.0 | 65.0 | 60.0 | 58.0 | 65.0 | Delta |
| | 2021 | | | | | | | | | | | | | | | | |
| VSAP29 | September | Saliva | 30.8 | 58.8 | 58.0 | 64.8 | 59.6 | 58.0 | 63.0 | Delta | 58.0 | 57.0 | 64.0 | 59.0 | 57.0 | 63.0 | Delta |
| | 2021 | | | | | | | | | | | | | | | | |
| VSAP30 | September | NA | 27.3 | 59.8 | 58.9 | 66.0 | 60.7 | 59.2 | 64.0 | Delta | 59.0 | 58.0 | 65.0 | 60.0 | 58.0 | 64.0 | Delta |
| | 2021 | | | | | | | | | | | | | | | | |
| VSAP31 | September | NP | 29.0 | 59.9 | 59.3 | 65.8 | 60.6 | 59.0 | 63.8 | Delta | 59.0 | 58.0 | 65.0 | 60.0 | 58.0 | 64.0 | Delta |
| | 2021 | | | | | | | | | | | | | | | | |
| VSAP32 | September | NP | 29.7 | 59.6 | 58.9 | 65.9 | 60.7 | 59.1 | 63.9 | Delta | 59.0 | 58.0 | 65.0 | 59.5 | 58.0 | 64.0 | Delta |
| | 2021 | | | | | | | | | | | | | | | | |
| VSAP33 | September | NP | 24.9 | 60.2 | 59.2 | 65.8 | 60.8 | 59.2 | 64.1 | Delta | 0 | 0 | 65.0 | 59.0 | 57.0 | 64.0 | P- |
| | 2021 | | | | | | | | | | | | | | | | Delta |
| VSAP34 | October | NP | 26.9 | 60.6 | 58.9 | 65.7 | 60.5 | 59.1 | 63.9 | Delta | 59.0 | 58.0 | 65.0 | 59.0 | 58.0 | 64.0 | Delta |
| | 2021 | | | | | | | | | | | | | | | | |

| | | RGQ | | | | | | | ABI 7500 | | | | | | |
|---|---|---|---|---|---|---|---|---|---|---|---|---|---|---|---|
| | | SMB-501 | | SMB-484 | | SMB-452 | | | SMB-501 | | SMB-484 | | SMB-452 | | |
| | | Assay | | Assay | | Assay | | | Assay | | Assay | | Assay | | |
| Ref./Pt. | | WT | MT | WT | MT | WT | MT | Final | WT | MT | WT | MT | WT | MT | Final |
| samples | DOC | (Cy3) | (Cy5) | (Cy3) | (Cy5) | (Cy3) | (Cy5) | id | (Cy3) | (Cy5) | (Cy3) | (Cy5) | (Cy3) | (Cy5) | id |
| WT-Reference | | 61.1 | 59.3 | 66.6 | 60.7 | 64.1 | 59.5 | z | 59.4 | 57.9 | 64.9 | 59.8 | 62.1 | 58.9 | |
| MT-B.1.1.7 | | 55.6 | 63.2 | 66.6 | 60.7 | 64.1 | 59.5 | | 53.3 | 62.1 | 64.9 | 59.8 | 62.1 | 58.9 | |
| (Alpha) | | | | | | | | | | | | | | | |
| MT-B.1.351 | | 55.6 | 63.2 | 62.9 | 69.2 | 64.1 | 59.5 | | 53.3 | 62.1 | 63.3 | 64.2 | 62.1 | 58.9 | |
| (Beta) | | | | | | | | | | | | | | | |
| MT-B.1.617.2 | | 61.1 | 59.3 | 66.6 | 60.7 | 0 | 64.9 | | 59.4 | 57.9 | 64.9 | 59.8 | 57.5 | 62.9 | |
| (Delta) | | | | | | | | | | | | | | | |
| MT-B.1.529 | | | | | | | | | | | | | | | |
| (Omicron) | | | | | | | | | | | | | | | |
| VSAP1 | April | 61.5 | 0 | 66.7 | 61.0 | 63.8 | 60.5 | WT | 59.7 | 58.2 | 65.2 | 59.9 | 62.4 | 59.5 | WT |
| | 2021 | | | | | | | | | | | | | | |
| VSAP2 | April | 0 | 0 | 66.8 | 61.0 | 64.0 | 60.8 | Ind | 53.2 | 61.9 | 64.8 | 59.5 | 62.1 | 59.1 | Alpha |
| | 2021 | | | | | | | | | | | | | | |
| VSAP3 | May | 0 | 0 | 67.0 | 61.0 | 0 | 0 | Ind | 59.5 | 57.1 | 65.4 | 59.7 | 57.7 | 63.6 | Delta |
| | 2021 | | | | | | | | | | | | | | |
| VSAP4 | May | 0 | 0 | 66.8 | 61.0 | 64.3 | 0 | Ind | 53.6 | 62.1 | 65.2 | 59.7 | 62.3 | 59.1 | Alpha |
| | 2021 | | | | | | | | | | | | | | |
| VSAP5 | May | 61.3 | 59.3 | 66.7 | 61.0 | 59.8 | 64.7 | Delta | 59.5 | 58.0 | 65.2 | 59.7 | 58.0 | 63.5 | Delta |
| | 2021 | | | | | | | | | | | | | | |
| VSAP6 | May | 56.0 | 63.3 | 67.0 | 61.2 | 64.5 | 0 | Alpha | 53.7 | 62.3 | 65.4 | 59.9 | 62.6 | 0 | P- |
| | 2021 | | | | | | | | | | | | | | Alpha |
| VSAP7 | June | 0 | 0 | 66.8 | 61.0 | 59.7 | 64.7 | Ind | 59.1 | 58.6 | 63.0 | 59.6 | 58.0 | 63.4 | Delta |
| | 2021 | | | | | | | | | | | | | | |
| VSAP8 | July | 61.5 | 60.0 | 67.5 | 61.5 | 0 | 65.5 | Delta | 59.5 | 57.6 | 63.1 | 59.5 | 57.8 | 63.5 | Delta |
| | 2021 | | | | | | | | | | | | | | |
| VSAP9 | July | 60.7 | 59.5 | 67.0 | 61.2 | 60.0 | 64.9 | Delta | 59.5 | 58.0 | 65.0 | 59.6 | 57.8 | 63.4 | Delta |
| | 2021 | | | | | | | | | | | | | | |
| VSAP10 | July | 0 | 0 | 66.9 | 60.9 | 0 | 64.8 | Ind | 59.9 | 58.4 | 65.7 | 60.1 | 58.2 | 64.2 | Delta |
| | 2021 | | | | | | | | | | | | | | |
| VSAP11 | July | 61.2 | 59.7 | 67.2 | 61.4 | 59.8 | 65.0 | Delta | 59.2 | 57.3 | 65.3 | 59.9 | 58.3 | 63.5 | Delta |
| | 2021 | | | | | | | | | | | | | | |
| VSAP12 | July | 0 | 0 | 66.9 | 61.1 | 59.4 | 64.7 | Ind | 59.5 | 57.7 | 64.9 | 59.6 | 57.9 | 63.3 | Delta |
| | 2021 | | | | | | | | | | | | | | |
| VSAP13 | July | 0 | 0 | 66.8 | 61.0 | 59.3 | 64.8 | Delta | 59.1 | 57.8 | 64.9 | 59.5 | 57.8 | 63.4 | Delta |
| | 2021 | | | | | | | | | | | | | | |
| VSAP14 | July | 61.0 | 59.5 | 66.8 | 61.2 | 59.8 | 64.7 | Delta | 59.5 | 58.0 | 65.3 | 59.7 | 58.0 | 63.6 | Delta |
| | 2021 | | | | | | | | | | | | | | |
| VSAP15 | July | 61.0 | 59.3 | 66.8 | 61.0 | 59.5 | 64.7 | Delta | 59.5 | 57.8 | 65.1 | 59.7 | 58.0 | 63.6 | Delta |
| | 2021 | | | | | | | | | | | | | | |
| VSAP16 | July | 0 | 0 | 66.8 | 61.2 | 59.7 | 64.8 | Ind | 59.3 | 58.6 | 65.1 | 59.7 | 58.2 | 63.4 | Delta |
| | 2021 | | | | | | | | | | | | | | |
| VSAP17 | July | 0 | 0 | 0 | 0 | 0 | 0 | Neg | 0 | 0 | 0 | 0 | 0 | 0 | Neg |
| | 22021 | | | | | | | | | | | | | | |
| VSAP18 | July | 61.4 | 59.7 | 67.0 | 61.3 | 59.8 | 65.0 | Delta | 59.5 | 58.4 | 65.2 | 59.8 | 58.2 | 63.6 | Delta |
| | 2021 | | | | | | | | | | | | | | |
| VSAP19 | July | 0 | 0 | 66.7 | 61.0 | 59.2 | 64.7 | Ind | 59.5 | 57.8 | 65.2 | 59.5 | 57.9 | 63.3 | Delta |
| | 2021 | | | | | | | | | | | | | | |
| VSAP20 | August | 60.8 | 59.3 | 66.5 | 61.0 | 59.7 | 64.5 | Delta | 59.1 | 58.4 | 65.0 | 59.7 | 57.8 | 63.1 | Delta |
| | 2021 | | | | | | | | | | | | | | |
| VSAP21 | August | 58.7 | 57.3 | 64.5 | 58.8 | 57.5 | 62.5 | Delta | 59.7 | 58.4 | 65.5 | 60.0 | 58.2 | 63.9 | Delta |
| | 2021 | | | | | | | | | | | | | | |
| VSAP22 | August | 0 | 0 | 66.7 | 60.5 | 59.5 | 64.5 | Ind | 59.3 | 57.8 | 65.0 | 59.5 | 57.8 | 63.3 | Delta |
| | 2021 | | | | | | | | | | | | | | |
| VSAP23 | August | 62.0 | 60.2 | 67.5 | 61.7 | 60.0 | 65.5 | Delta | 59.7 | 58.6 | 65.5 | 60.0 | 58.2 | 63.3 | Delta |
| | 2021 | | | | | | | | | | | | | | |
| VSAP24 | August | 0 | 0 | 66.5 | 0 | 0 | 0 | Ind | 0 | 0 | 65.1 | 0 | 0 | 63.3 | Ind |
| | 2021 | | | | | | | | | | | | | | |
| VSAP25 | August | 61.3 | 59.7 | 67.0 | 61.3 | 60.0 | 64.9 | Delta | 59.3 | 58.0 | 65.2 | 59.5 | 58.0 | 63.5 | Delta |
| | 2021 | | | | | | | | | | | | | | |
| VSAP26 | August | 62.0 | 59.7 | 0 | 0 | 59.7 | 64.5 | P- | 60.0 | 57.6 | 65.8 | 59.9 | 0 | 64.4 | P- |
| | 2021 | | | | | | | Delta | | | | | | | Delta |
| VSAP27 | September | 61.0 | 59.7 | 67.0 | 61.5 | 60.0 | 65.0 | Delta | 59.3 | 58.2 | 65.2 | 59.9 | 58.2 | 63.7 | Delta |
| | 2021 | | | | | | | | | | | | | | |
| VSAP28 | September | 61.0 | 59.5 | 66.7 | 61.0 | 59.7 | 64.5 | Delta | 59.3 | 58.0 | 65.2 | 59.9 | 58.0 | 63.5 | Delta |
| | 2021 | | | | | | | | | | | | | | |
| VSAP29 | September | 59.7 | 58.5 | 65.7 | 60.0 | 58.8 | 63.5 | Delta | 58.3 | 57.7 | 64.0 | 58.8 | 57.1 | 62.3 | Delta |
| | 2021 | | | | | | | | | | | | | | |
| VSAP30 | September | 61.2 | 59.8 | 67.2 | 61.5 | 60.4 | 65.1 | Delta | 59.7 | 58.7 | 65.5 | 60.0 | 58.2 | 63.9 | Delta |
| | 2021 | | | | | | | | | | | | | | |
| VSAP31 | September | 61.0 | 59.5 | 67.0 | 61.4 | 60.1 | 65.0 | Delta | 59.3 | 58.2 | 65.2 | 59.3 | 58.2 | 63.5 | Delta |
| | 2021 | | | | | | | | | | | | | | |
| VSAP32 | September | 61.3 | 59.3 | 67.3 | 61.5 | 60.4 | 65.2 | Delta | 59.5 | 58.2 | 65.3 | 60.0 | 58.2 | 63.7 | Delta |
| | 2021 | | | | | | | | | | | | | | |
| VSAP33 | September | 0 | 0 | 67.0 | 61.0 | 59.7 | 0 | Ind | 59.3 | 57.7 | 65.0 | 59.5 | 57.7 | 63.3 | Delta |
| | 2021 | | | | | | | | | | | | | | |
| VSAP34 | October | 61.3 | 59.5 | 66.7 | 61.0 | 59.5 | 64.5 | Delta | 59.5 | 58.0 | 65.0 | 59.7 | 57.8 | 63.1 | Delta |
| | 2021 | | | | | | | | | | | | | | |

The Tm values from patient specimens were identified using the VOC identification macro. Samples until VSAP34 were tested in all 4 RT-PCR instruments and Ref-Reference strains;
IC—Internal Control;
Ind—indeterminate (>2 probe failures);
Neg—Negative;
Id—Identification;
WT—Wild type;
MT—Mutant;
P—Presumptive;

TABLE 5
Confirmation of mutations in patient samples by sequencing.

| | | Confirmation by Sanger sequencing | | | |
|---|---|---|---|---|---|
| | | | 484 probe | 452 probe | |
| | | 501 probe | binding | binding | |
| Reference | | binding | region | region | |
| strains/ | | region | (Aminoacid | (Aminoacid | |
| Patient | SMB genotyping | (Aminoacid | and | and | |
| samples | assay Id | and position) | position) | position) | Sequencing id |
| USA-WA1/2020 (WT) | | AAT (N501) | GAA (E484) | CTG (L) | WT |
| B.1.1.7 (Alpha) | | TAT (Y501) | GAA (E484) | CTG (L) | B.1.1.7 (Alpha) |
| B.1.351 (Beta) | | TAT (Y501) | AAA (K484) | CTG (L) | B.1.351 (Beta) |
| B.1.617.2 (Delta) | | AAT (N501) | GAA (E484) | CGG (R) | B.1.617.2 (Delta) |
| B.1.529 (Omicron BA.1) | | AGT (S496); CGA | GCA (A484) | CTG (L) | B.1.529 |
| | | (R498); | | | (Omicron BA.1) |
| | | TAT(Y501); CAC | | | |
| | | (H505) | | | |
| B.1.529 (Omicron BA.2) | | CGA (R498); TAT | GCA (A484) | CTG (L) | B.1.529 |
| | | (Y501); CAC | | | (Omicron BA.2) |
| | | (H505) | | | |
| B.1.529 (Omicron BA.2.12.1) | | CGA (R498); TAT | GCA (A484) | CAG (Q) | B.1.529 |
| | | (Y501); CAC | | | (Omicron |
| | | (H505) | | | BA.2.12.1) |
| B.1.529 (Omicron BA.4/5) | | CGA (R498); TAT | GCA (A484); | CGG (R) | B.1.529 |
| | | (Y501); CAC | GTT (F486V) | | (Omicron |
| | | (H505) | | | BA.4/5) |
| VSAP1 | WT | AAT (N) | GAA (E) | CTG (L) | WT |
| VSAP2 | B.1.1.7 (Alpha) | TAT (Y) | GAA (E) | CTG (L) | B.1.1.7 (Alpha) |
| VSAP3 | B.1.617.2 (Delta) | AAT (N) | GAA (E) | CGG (R) | B.1.617.2 (Delta) |
| VSAP4 | B.1.1.7 (Alpha) | TAT (Y) | GAA (E) | CTG (L) | B.1.1.7 (Alpha) |
| VSAP5 | B.1.617.2 (Delta) | AAT (N) | GAA (E) | CGG (R) | B.1.617.2 (Delta) |
| VSAP6 | B.1.1.7 (Alpha) | TAT (Y) | GAA (E) | CTG (L) | B.1.1.7 (Alpha) |
| VSAP7 | B.1.617.2 (Delta) | AAT (N) | GAA (E) | CGG (R) | B.1.617.2 (Delta) |
| VSAP8 | B.1.617.2 (Delta) | AAT (N) | GAA (E) | CGG (R) | B.1.617.2 (Delta) |
| VSAP9 | B.1.617.2 (Delta) | AAT (N) | GAA (E) | CGG (R) | B.1.617.2 (Delta) |
| VSAP11 | B.1.617.2 (Delta) | AAT (N) | GAA (E) | CGG (R) | B.1.617.2 (Delta) |
| VSAP12 | B.1.617.2 (Delta) | AAT (N) | GAA (E) | CGG (R) | B.1.617.2 (Delta) |
| VSAP13 | B.1.617.2 (Delta) | AAT (N) | GAA (E) | CGG (R) | B.1.617.2 (Delta) |
| VSAP14 | B.1.617.2 (Delta) | AAT (N) | GAA (E) | CGG (R) | B.1.617.2 (Delta) |
| VSAP15 | B.1.617.2 (Delta) | AAT (N) | GAA (E) | CGG (R) | B.1.617.2 (Delta) |
| VSAP16 | B.1.617.2 (Delta) | AAT (N) | GAA (E) | CGG (R) | B.1.617.2 (Delta) |
| VSAP17 | Negative | | | | ND |
| VSAP18 | B.1.617.2 (Delta) | AAT (N) | GAA (E) | CGG (R) | B.1.617.2 (Delta) |
| VSAP19 | B.1.617.2 (Delta) | AAT (N) | GAA (E) | CGG (R) | B.1.617.2 (Delta) |
| VSAP20 | B.1.617.2 (Delta) | AAT (N) | GAA (E) | CGG (R) | B.1.617.2 (Delta) |
| VSAP21 | B.1.617.2 (Delta) | AAT (N) | GAA (E) | CGG (R) | B.1.617.2 (Delta) |
| VSAP22 | B.1.617.2 (Delta) | AAT (N) | GAA (E) | CGG (R) | B.1.617.2 (Delta) |
| VSAP23 | B.1.617.2 (Delta) | AAT (N) | GAA (E) | CGG (R) | B.1.617.2 (Delta) |
| VSAP24 | B.1.617.2 (Delta) | AAT (N) | GAA (E) | CGG (R) | B.1.617.2 (Delta) |
| VSAP25 | B.1.617.2 (Delta) | AAT (N) | GAA (E) | CGG (R) | B.1.617.2 (Delta) |
| VSAP26 | B.1.617.2 (Delta) | AAT (N) | GAA (E) | CGG (R) | B.1.617.2 (Delta) |
| VSAP27 | B.1.617.2 (Delta) | AAT (N) | GAA (E) | CGG (R) | B.1.617.2 (Delta) |
| VSAP28 | B.1.617.2 (Delta) | AAT (N) | GAA (E) | CGG (R) | B.1.617.2 (Delta) |
| VSAP29 | B.1.617.2 (Delta) | AAT (N) | GAA (E) | CGG (R) | B.1.617.2 (Delta) |
| VSAP30 | B.1.617.2 (Delta) | AAT (N) | GAA (E) | CGG (R) | B.1.617.2 (Delta) |
| VSAP31 | B.1.617.2 (Delta) | AAT (N) | GAA (E) | CGG (R) | B.1.617.2 (Delta) |
| VSAP32 | B.1.617.2 (Delta) | AAT (N) | GAA (E) | CGG (R) | B.1.617.2 (Delta) |
| VSAP33 | B.1.617.2 (Delta) | AAT (N) | GAA (E) | CGG (R) | B.1.617.2 (Delta) |
| VSAP34 | B.1.617.2 (Delta) | AAT (N) | GAA (E) | CGG (R) | B.1.617.2 (Delta) |
| VSAP35 | B.1.617.2 (Delta) | AAT (N) | GAA (E) | CGG (R) | B.1.617.2 (Delta) |
| VSAP36 | VARIANT | AAT (N) | GAA (E) | CGG (R) | B.1.617.2 (Delta) |
| | INDETERMINATE | | | | |
| VSAP37 | B.1.617.2 (Delta) | AAT (N) | GAA (E) | CGG (R) | B.1.617.2 (Delta) |
| VSAP38 | VARIANT | AAT (N) | GAA (E) | CGG (R) | B.1.617.2 (Delta) |
| | INDETERMINATE | | | | |
| VSAP39 | VARIANT | AAT (N) | GAA (E) | CGG (R) | B.1.617.2 (Delta) |
| | INDETERMINATE | | | | |
| VSAP40 | Pres B.1.617.2 | AAT (N) | GAA (E) | CGG (R) | B.1.617.2 (Delta) |
| | (Delta) | | | | |
| VSAP41 | Pres B.1.617.2 | AAT (N) | GAA (E) | CGG (R) | B.1.617.2 (Delta) |
| | (Delta) | | | | |
| VSAP42 | B.1.617.2 (Delta) | AAT (N) | GAA (E) | CGG (R) | B.1.617.2 (Delta) |
| VSAP43 | VARIANT | AAT (N) | GAA (E) | CGG (R) | B.1.617.2 (Delta) |
| | INDETERMINATE | | | | |
| VSAP44 | B.1.617.2 (Delta) | AAT (N) | GAA (E) | CGG (R) | B.1.617.2 (Delta) |
| VSAP45 | B.1.617.2 (Delta) | AAT (N) | GAA (E) | CGG (R) | B.1.617.2 (Delta) |
| VSAP46 | B.1.617.2 (Delta) | AAT (N) | GAA (E) | CGG (R) | B.1.617.2 (Delta) |
| VSAP47 | Pres B.1.617.2 | AAT (N) | GAA (E) | CGG (R) | B.1.617.2 (Delta) |
| | (Delta) | | | | |
| VSAP48 | B.1.617.2 (Delta) | AAT (N) | GAA (E) | CGG (R) | B.1.617.2 (Delta) |
| VSAP49 | B.1.617.2 (Delta) | AAT (N) | GAA (E) | CGG (R) | B.1.617.2 (Delta) |
| VSAP50 | B.1.617.2 (Delta) | AAT (N) | GAA (E) | CGG (R) | B.1.617.2 (Delta) |
| VSAP51 | B.1.617.2 (Delta) | AAT (N) | GAA (E) | CGG (R) | B.1.617.2 (Delta) |
| VSAP52 | B.1.617.2 (Delta) | AAT (N) | GAA (E) | CGG (R) | B.1.617.2 (Delta) |
| VSAP53 | B.1.617.2 (Delta) | AAT (N) | GAA (E) | CGG (R) | B.1.617.2 (Delta) |
| VSAP54 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP55 | B.1.617.2 (Delta) | AAT (N) | GAA (E) | CGG (R) | B.1.617.2 (Delta) |
| VSAP56 | Pres B.1.529 | AGT (S); CGA (R); | GCA (A) | CTG (L) | B.1.529 |
| | (Omicron BA.1) | TAT (Y); CAC (H) | | | (Omicron BA.1) |
| VSAP57 | B.1.617.2 (Delta) | AAT (N) | GAA (E) | CGG (R) | B.1.617.2 (Delta) |
| VSAP58 | B.1.617.2 (Delta) | AAT (N) | GAA (E) | CGG (R) | B.1.617.2 (Delta) |
| VSAP59 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP60 | B.1.617.2 (Delta) | AAT (N) | GAA (E) | CGG (R) | B.1.617.2 (Delta) |
| VSAP61 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP62 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP63 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP64 | B.1.617.2 (Delta) | AAT (N) | GAA (E) | CGG (R) | B.1.617.2 (Delta) |
| VSAP65 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP66 | B.1.617.2 (Delta) | AAT (N) | GAA (E) | CGG (R) | B.1.617.2 (Delta) |
| VSAP67 | B.1.617.2 (Delta) | AAT (N) | GAA (E) | CGG (R) | B.1.617.2 (Delta) |
| VSAP68 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP69 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP70 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP71 | Pres B.1.529 | AGT (S); CGA (R); | GCA (A) | CTG (L) | B.1.529 |
| | (Omicron BA.1) | TAT (Y); CAC (H) | | | (Omicron BA.1) |
| VSAP72 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP73 | B.1.617.2 (Delta) | AAT (N) | GAA (E) | CGG (R) | B.1.617.2 (Delta) |
| VSAP74 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP75 | B.1.529 (Omicron | CGA (R); TAT (Y); | GCA (A); | CGG (R) | B.1.529 |
| | BA.4/5) | CAC (H) | GTT (V) | | (Omicron |
| | | | | | BA.5.2)^{a} |
| VSAP76 | B.1.529 (Omicron | AGT (S); CGA (R); | GCA (A) | CTG (L) | B.1.529 |
| | BA.1) | TAT (Y); CAC (H) | | | (Omicron |
| | | | | | BA.1.1)^{a} |
| VSAP77 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP78 | B.1.529 (Omicron | AGT (S); CGA (R); | GCA (A) | CTG (L) | B.1.529 |
| | BA.1) | TAT (Y); CAC (H) | | | (Omicron BA.1) |
| VSAP79 | VARIANT | CAA (Q, WT); | GYT (?), | CGG (R) | Inconclusive*^{a} |
| | INDETERMINATE | AAT (N, WT); | GAA (E) | | |
| | | TAC (Y, WT) | | | |
| VSAP80 | B.1.529 (Omicron) | CGA (R); TAT (Y); | GCA (A) | CTG (L) | B.1.529 |
| | BA.2 | CAC (H | | | (Omicron BA.2)^{a} |
| VSAP81 | B.1.529 (Omicron | CGA (R); TAT (Y); | GCA (A) | CTG (L) | B.1.529 |
| | BA.2) | CAC (H) | | | (Omicron BA.2) |
| VSAP82 | B.1.529 (Omicron | CGA (R); TAT (Y); | GCA (A) | CTG (L) | B.1.529 |
| | BA.2) | CAC (H) | | | (Omicron BA.2) |
| VSAP83 | Pres B.1.529 | CGA (R); TAT (Y); | GCA (A) | CTG (L) | B.1.529 |
| | (Omicron BA.2) | CAC (H) | | | (Omicron BA.2) |
| VSAP84 | B.1.529 (Omicron | CGA (R); TAT (Y); | GCA (A); | CGG (R) | B.1.529 |
| | BA.4/5) | CAC (H) | GTT (V) | | (Omicron |
| | | | | | BA.4.2)^{a} |
| VSAP85 | B.1.529 (Omicron | CGA (R); TAT (Y); | GCA (A) | CAG (Q) | B.1.529 |
| | BA.2.12.1) | CAC (H) | | | (Omicron |
| | | | | | BA.2.12.1)^{a} |
| VSAP86 | B.1.529 (Omicron | CGA (R); TAT (Y); | GCA (A) | CAG (Q) | B.1.529 |
| | BA.2.12.1) | CAC (H) | | | (Omicron |
| | | | | | BA.2.12.1) |
| VSAP87 | B.1.529 (Omicron | CGA (R); TAT (Y); | GCA (A) | CAG (Q) | B.1.529 |
| | BA.2.12.1) | CAC (H) | | | (Omicron |
| | | | | | BA.2.12.1) |
| VSAP88 | B.1.529 (Omicron | CGA (R); TAT (Y); | GCA (A) | CTG (L) | B.1.529 |
| | BA.2) | CAC (H) | | | (Omicron BA.2) |
| VSAP89 | B.1.529 (Omicron | CGA (R); TAT (Y); | GCA (A) | CTG (L) | B.1.529 |
| | BA.2) | CAC (H) | | | (Omicron BA.2) |
| VSAP90 | B.1.529 (Omicron | CGA (R); TAT (Y); | GCA (A); | CGG (R) | B.1.529 |
| | BA.4/5) | CAC (H) | GTT (V) | | (Omicron |
| | | | | | BA.5.5)^{a} |
| VSAP91 | B.1.529 (Omicron | CGA (R); TAT (Y); | GCA (A); | CGG (R) | B.1.529 |
| | BA.4/5) | CAC (H) | GTT (V) | | (Omicron |
| | | | | | BA.4/5) |

*Sanger sequencing showed inconclusive mutation (GAA-GYA) at the 484 codon. Y = C/T; if GAA-GTA it would be WT, if GAA-GCA it is an undefined mutation that does not match with the reference sequences. However, the strain was confirmed as Delta AY.25.1 by whole genome sequencing.
^{a}confirmed by whole genome sequencing
Pres.: Presumptive (presumptive call is made when one or more probes in the assay fails to give a Tm)
ND: not done.

TABLE 6
Sensitivity and specificity of SMB-VOC assay compared to sequencing.

| | | Sequencing | | | | | | | | | |
|---|---|---|---|---|---|---|---|---|---|---|---|
| | CoV-2 | | | | Omicron | Omicron | Omicron | Omicron | | | Combined |
| | Variants | WT | Alpha | Delta | BA.1 | BA.2 | BA.2.12.1 | BA.4/5 | ND | Neg | % Sensitivity |
| SMB- | WT | 1 | | | | | | | | | |
| VOC assay | Alpha | | 3 | | | | | | | | |
| | Delta | | | 51 | | | | | | | |
| | Omicron | | | | 16 | | | | | | |
| | BA.1 | | | | | | | | | | |
| | Omicron | | | | | 6 | | | | | |
| | BA.2 | | | | | | | | | | |
| | Omicron | | | | | | 3 | | | | |
| | BA.2.12.1 | | | | | | | | | | |
| | Omicron | | | | | | | 3 | 1 | | |
| | BA.4/5 | | | | | | | | | | |
| | Ind | | | 5 | | | | | | | |
| | ND | | | | | | | | 1 | | |
| | Neg | | | | | | | | | 9 | |
| % Sensitivity | | 100 | 100 | 91 | 100 | 100 | 100 | 100 | 100 | | 94.3 |
| Combined | | 100 | | | | | | | | | |
| % Specificity | | | | | | | | | | | |

Sensitivity = TP/(TP + FN) and Specificity = TN/(FP + TN), where TP = True positives; TN = True negatives; FN = False negatives.

TABLE 7A
Exemplary Excel Analyze Tool
Table A

| | | | 501 - WT | 501 - MT | 484 - WT | 484 - MT | 452 - WT | 452 - MT |
|---|---|---|---|---|---|---|---|---|
| | Interna | CoV-N | probe | probe | probe | probe | probe | probe |
| | Control | probe | Tm 1 | Tm 2 | Tm 3 | Tm 4 | Tm 5 | Tm 6 |
| | Ct | Ct | (° C.) | (° C.) | (° C.) | (° C.) | (° C.) | (° C.) |
| INPUT Ct's & | 25.2 | 28.2 | 50.37 | 58.66 | 58.37 | 55.37 | 58.92 | 63.75 |
| Tm's -- > | | | | | | | | |

| | | | |
|---|---|---|---|
| | SARS-CoV-2 | B.1.529 | |
| Result | POSITIVE | (Omicron BA.4/5) | 0.38 |

ANALYZE DATA

TABLE 7B
Exemplary Excel Analyze Tool (continued)
Table B (Updated by Worksheet)

| | 501 - WT | 501 - MT | 484 - WT | 484 - MT | 452 - WT | 452 - MT | | |
|---|---|---|---|---|---|---|---|---|
| Unknown | probe | probe | probe | probe | probe | probe | Internal | CoV-N |
| Species/ | Tm 1 | Tm 2 | Tm 3 | Tm 4 | Tm 5 | Tm 6 | Control | probe |
| Sample | (° C.) | (° C.) | (° C.) | (° C.) | (° C.) | (° C.) | Ct | Ct |
| INPUT | 50.37 | 58.66 | 58.37 | 55.37 | 58.92 | 63.75 | 25.20 | 28.20 |
| Tm's --> | | | | | | | | |
| Match | Species 1 | | D-value | | | | | |
| Deter- | B.1.529 | | 0.383927076 | | | | | |
| mined by | (Omicron BA.4/5) | | | | | | | |
| SQRT | | | | | | | | |
| Sum((Delta | | | | | | | | |

Tm)\hat{ }2)
Method

TABLE 7C
Exemplary Excel Analyze Tool (continued)
Table C (Updated by User)

| | 501 - WT | 501 - MT | 484 - WT | 484 - MT | 452 - WT | 452 - MT |
|---|---|---|---|---|---|---|
| | probe | probe | probe | probe | probe | probe |
| Calibrator Species | Tm 1 (° C.) | Tm 2 (° C.) | Tm 3 (° C.) | Tm 4 (° C.) | Tm 5 (° C.) | Tm 6 (° C.) |
| Wild type (Ancestral) | 59.70 | 59.00 | 65.70 | 60.50 | 61.90 | 59.90 |
| B.1.1.7 (Alpha) | 55.70 | 58.90 | 65.70 | 60.60 | 61.90 | 59.90 |
| B.1.351 (Beta) | 55.70 | 58.90 | 62.20 | 64.70 | 61.90 | 59.90 |
| B.1.617.2 (Delta) | 59.70 | 58.90 | 65.70 | 60.60 | 58.30 | 63.20 |
| B.1.529 (Omicron BA1) | 48.70 | 56.30 | 63.60 | 60.60 | 61.90 | 59.90 |
| Presumptive B.1.617.2 | 59.70 | 58.90 | 65.70 | 0.00 | 58.30 | 63.20 |
| (Delta) | | | | | | |
| Presumptive B.1.617.2 | 59.70 | 58.90 | 0.00 | 0.00 | 58.30 | 63.20 |
| (Delta) | | | | | | |
| Presumptive B.1.617.2 | 0.00 | 0.00 | 65.70 | 60.60 | 58.30 | 63.20 |
| (Delta) | | | | | | |
| Presumptive B.1.617.2 | 59.70 | 59.00 | 65.70 | 0.00 | 58.30 | 63.20 |
| (Delta) | | | | | | |
| Presumptive B.1.529 | 0.00 | 56.30 | 63.60 | 60.60 | 61.90 | 59.90 |
| (Omicron) | | | | | | |
| Presumptive B.1.617.2 | 59.70 | 59.00 | 65.70 | 60.60 | 58.30 | 0.00 |
| (Delta | | | | | | |
| Presumptive B.1.529 | 48.97 | 56.66 | 0.00 | 0.00 | 62.29 | 59.25 |
| (Omicron) | | | | | | |
| Presumptive B.1.529 | 48.52 | 56.21 | 63.70 | 0.00 | 61.77 | 58.67 |
| (Omicron) | | | | | | |
| Presumptive B.1.617.2 | 54.00 | 62.00 | 65.00 | 59.00 | 62.00 | 0.00 |
| (Delta) | | | | | | |
| Wild type (Ancestral) | 59.00 | 58.00 | 65.00 | 59.80 | 62.00 | 60.00 |
| B.1.1.7 (Alpha) | 54.50 | 62.00 | 65.00 | 59.80 | 62.00 | 60.00 |
| B.1.351 (Beta) | 54.50 | 62.00 | 61.00 | 64.00 | 62.00 | 60.00 |
| B.1.617.2 (Delta) | 59.00 | 58.00 | 65.00 | 59.80 | 58.00 | 64.00 |
| Presumptive B.1.617.2 | 59.00 | 58.00 | 65.00 | 60.00 | 0.00 | 64.00 |
| (Delta) | | | | | | |
| Presumptive B.1.529 | 48.52 | 56.21 | 64.00 | 59.00 | 0.00 | 0.00 |
| (Omicron BA.1) | | | | | | |
| Presumptive B.1.529 | 48.70 | 56.30 | 63.60 | 60.60 | 61.90 | 0.00 |
| (Omicron BA.1) | | | | | | |
| Presumptive B.1.529 | 48.52 | 56.21 | 0.00 | 0.00 | 61.77 | 0.00 |
| (Omicron BA.1) | | | | | | |
| Presumptive B.1.617.2 | 0.00 | 58.71 | 65.55 | 60.39 | 58.73 | 63.49 |
| (Delta) | | | | | | |
| B.1.529 (Omicron BA.1) | 48.74 | 56.58 | 62.72 | 59.97 | 62.54 | 58.95 |
| B.1.529 (Omicron BA.2) | 50.51 | 58.58 | 63.02 | 60.22 | 62.64 | 60.1 |
| Presumptive B.1.529 | | 58.58 | 63.02 | 60.22 | 62.64 | 60.1 |
| (Omicron BA.2) | | | | | | |
| B.1.529 (Omicron | 50.80 | 58.81 | 62.98 | 60.29 | 60.14 | 61.27 |
| BA.2.12.1) | | | | | | |
| B.1.529 (Omicron BA.4/5) | 50.60 | 58.62 | 58.21 | 55.13 | 58.88 | 63.84 |

TABLE 7D
Exemplary Excel Analyze Tool (continued)
Table D (Updated by Worksheet - Do Not Modify)

| | 501 - | 501 - | 484 - | 484 - | 452 - | 452 - | |
|---|---|---|---|---|---|---|---|
| | WT probe | MT probe | WT probe | MT probe | WT probe | MT probe | |
| | SQR | SQR | SQR | SQR | SQR | SQR | SQRT |
| | (Delta | (Delta | (Delta | (Delta | (Delta | (Delta | Sum |
| Calibrator Species | Tm 1) | Tm 2) | Tm 3) | Tm 4) | Tm 5) | Tm 6) | (Deltas) |
| Wild type (Ancestral) | 87.05 | 0.12 | 53.73 | 26.32 | 8.88 | 14.82 | 13.82 |
| B.1.1.7 (Alpha) | 28.41 | 0.06 | 53.73 | 27.35 | 8.88 | 14.82 | 11.54 |
| B.1.351 (Beta) | 28.41 | 0.06 | 14.67 | 87.05 | 8.88 | 14.82 | 12.41 |
| B.1.617.2 (Delta) | 87.05 | 0.06 | 53.73 | 27.35 | 0.38 | 0.30 | 13.00 |
| B.1.529 (Omicron | 2.79 | 5.57 | 27.35 | 27.35 | 8.88 | 14.82 | 9.31 |
| BA1) | | | | | | | |
| Presumptive | 87.05 | 0.06 | 53.73 | 3065.84 | 0.38 | 0.30 | 56.63 |
| B.1.617.2 (Delta) | | | | | | | |
| Presumptive | 87.05 | 0.06 | 3407.06 | 3065.84 | 0.38 | 0.30 | 81.00 |
| B.1.617.2 (Delta) | | | | | | | |
| Presumptive | 2537.14 | 3441.00 | 53.73 | 27.35 | 0.38 | 0.30 | 77.85 |
| B.1.617.2 (Delta) | | | | | | | |
| Presumptive | 87.05 | 0.12 | 53.73 | 3065.84 | 0.38 | 0.30 | 56.63 |
| B.1.617.2 (Delta) | | | | | | | |
| Presumptive B.1.529 | 2537.14 | 5.57 | 27.35 | 27.35 | 8.88 | 14.82 | 51.20 |
| (Omicron) | | | | | | | |
| Presumptive | 87.05 | 0.12 | 53.73 | 27.35 | 0.38 | 4064.06 | 65.06 |
| B.1.617.2 (Delta) | | | | | | | |
| Presumptive B.1.529 | 1.96 | 4.00 | 3407.06 | 3065.84 | 11.36 | 20.25 | 80.69 |
| (Omicron) | | | | | | | |
| Presumptive B.1.529 | 3.42 | 6.00 | 28.41 | 3065.84 | 8.12 | 25.81 | 56.01 |
| (Omicron) | | | | | | | |
| Presumptive | 13.18 | 11.16 | 43.96 | 13.18 | 9.49 | 4064.06 | 64.46 |
| B.1.617.2 (Delta) | | | | | | | |
| Wild type (Ancestral) | 74.48 | 0.44 | 43.96 | 19.62 | 9.49 | 14.06 | 12.73 |
| B.1.1.7 (Alpha) | 17.06 | 11.16 | 43.96 | 19.62 | 9.49 | 14.06 | 10.74 |
| B.1.351 (Beta) | 17.06 | 11.16 | 6.92 | 74.48 | 9.49 | 14.06 | 11.54 |
| B.1.617.2 (Delta) | 74.48 | 0.44 | 43.96 | 19.62 | 0.85 | 0.06 | 11.81 |
| Presumptive | 74.48 | 0.44 | 43.96 | 21.44 | 3471.57 | 0.06 | 60.10 |
| B.1.617.2 (Delta) | | | | | | | |
| Presumptive B.1.529 | 3.42 | 6.00 | 31.70 | 13.18 | 3471.57 | 4064.06 | 87.12 |
| (Omicron BA. 1) | | | | | | | |
| Presumptive B.1.529 | 2.79 | 5.57 | 27.35 | 27.35 | 8.88 | 4064.06 | 64.31 |
| (Omicron BA.1) | | | | | | | |
| Presumptive B.1.529 | 3.42 | 6.00 | 3407.06 | 3065.84 | 8.12 | 4064.06 | 102.74 |
| (Omicron BA.1) | | | | | | | |
| Presumptive | 2537.14 | 0.00 | 51.55 | 25.20 | 0.04 | 0.07 | 51.13 |
| B.1.617.2 (Delta) | | | | | | | |
| B.1.529 (Omicron | 2.66 | 4.33 | 18.92 | 21.16 | 13.10 | 23.04 | 9.12 |
| BA.1) | | | | | | | |
| B.1.529 (Omicron | 0.02 | 0.01 | 21.62 | 23.52 | 13.84 | 13.32 | 8.50 |
| BA.2) | | | | | | | |
| Presumptive B.1.529 | 2537.14 | 0.01 | 21.62 | 23.52 | 13.84 | 13.32 | 51.08 |
| (Omicron BA.2) | | | | | | | |
| B.1.529 (Omicron | 0.18 | 0.02 | 21.25 | 24.21 | 1.49 | 6.15 | 7.30 |
| BA.2.12.1) | | | | | | | |
| B.1.529 (Omicron | 0.05 | 0.00 | 0.03 | 0.06 | 0.00 | 0.01 | 0.38 |
| BA.4/5) | | | | | | | |

What is claimed is:

1. A method for identifying a variant of a virus, comprising:
amplifying a viral nucleic acid of a variant of a virus in a sample with one or more primer pairs to obtain one or more amplicons, wherein each of the one or more primer pairs comprises a forward primer and a reverse primer, wherein the one or more primer pairs each specific for a target region of the viral nucleic acid, and wherein the one or more amplicons respectively correspond to one or more target regions of the viral nucleic acid;
contacting the one or more amplicons with one or more probes under a condition conducive to a hybridization reaction to form one or more probe-amplicon hybrids;
determining a melting temperature (Tm) of each of the one or more probe-amplicon hybrids;
determining a difference between the melting temperature of each of the one or more probe-amplicon hybrids and a reference melting temperature corresponding to the same probe-amplicon hybrid;
determining an aggregated difference of melting temperatures of the one or more probe-amplicon hybrids based on the difference between the melting temperature of each of the one or more probe-amplicon hybrids and the reference melting temperature corresponding to the same probe-amplicon hybrid; and
identifying the variant of the virus in the sample as a candidate variant, if the aggregated difference of the melting temperatures of the one or more probe-amplicon hybrids is identical to a reference aggregated difference of the candidate variant or if a difference between the aggregated difference of the melting temperatures of the one or more probe-amplicon hybrids and the reference aggregated difference of the candidate variant is less than a threshold value.

2. The method of claim 1, wherein the variant is a SARS-CoV-2 variant.

3. The method of claim 2, wherein the SARS-CoV-2 variant is selected from B.1.1.7 (20I, Alpha), B.1.351 (20H, Beta), P.1 (20J, Gamma), B.1.617.2 (21A, Delta), and B.1.1.529 (21K, Omicron) and the respective subvariants of Omicron.

4. The method of claim 1, wherein the step of amplifying is performed by a polymerase chain reaction (PCR).

5. The method of claim 1, wherein the sample comprises a viral genomic RNA or fragment thereof, and wherein prior to the step of amplifying, the method comprises a reverse transcription step by a reverse transcription-polymerase chain reaction (RT-PCR).

6. The method of claim 5, comprising extracting the viral genomic RNA or fragment thereof from the sample.

7. The method of claim 5, wherein the step of amplifying, the reverse transcription step, and the step of contacting are performed in a single reaction mixture.

8. The method of claim 1, wherein the step of amplifying for each of the one or more primer pairs is performed in separate reaction mixtures.

9. The method of claim 1, wherein at least one of the one or more target regions comprises one or more mutations characteristic of the variant of the virus.

10. The method of claim 2, wherein the one or more target regions comprise a codon of L452 or a substitution thereof, a codon of E484 or a substitution thereof, a codon of N501 or a substitution thereof, or a combination thereof.

11. The method of claim 10, wherein the one or more target regions comprise a codon of L452 or a substitution of L452Q or L452R, a codon of E484 or a substitution of E484K or E484A, a codon of N501 or a substitution of N501Y, or a combination thereof.

12. The method of claim 1, wherein the one or more primer pairs comprise a first primer pair capable of hybridizing to a first target region of the viral nucleic acid, a second primer pair capable of hybridizing to a second target region of the viral nucleic acid, and a third primer pair capable of hybridizing to a third target region of the viral nucleic acid.

13. The method of claim 12, wherein the first target region of the viral nucleic acid comprises a codon of L452 or a substitution of L452Q or L452R, the second target region of the viral nucleic acid comprises a codon of E484 or a substitution of E484K or E484A, and the third target region of the viral nucleic acid comprises a codon of N501 or a substitution of N501Y.

14. The method of claim 12, wherein the one or more amplicons comprise a first amplicon comprising the first target region, a second amplicon comprising the second target region, and a third amplicon comprising the third target region.

15. The method of claim 12, wherein the one or more probes comprise a first probe or a second probe capable of hybridizing to the first amplicon, a third probe or a fourth probe capable of hybridizing to the second amplicon, and a fifth probe or a sixth probe capable of hybridizing to the third amplicon.

16. The method of claim 12, wherein the first primer pairs comprise respective nucleotide sequences of SEQ ID NOs: 1-2, or comprise the respective nucleotide sequences having at least 90% sequence identity with the nucleotide sequences of SEQ ID NOs: 1-2; the second primer pairs comprise respective nucleotide sequences of SEQ ID NOs: 5-6 or SEQ ID NOs: 7-8; or comprise the respective nucleotide sequences having at least 90% sequence identity with the nucleotide sequences of SEQ ID NOs: 5-6 or SEQ ID NOs: 7-8; and/or the third primer pairs comprise respective nucleotide sequences of SEQ ID NOs: 11-12, or comprise the respective nucleotide sequences having at least 90% sequence identity with the nucleotide sequences of SEQ ID NOs: 11-12.

17. The method of claim 15, wherein the first probe comprises the nucleotide sequence of SEQ ID NO: 3, or comprises a nucleotide sequence having at least 90% sequence identity with the nucleotide sequence of SEQ ID NO: 3; and/or the second probe comprises the nucleotide sequence of SEQ ID NO: 4, or comprises a nucleotide sequence having at least 90% sequence identity with the nucleotide sequence of SEQ ID NO: 4.

18. The method of claim 15, wherein the third probe comprises the nucleotide sequence of SEQ ID NO: 9, or comprises a nucleotide sequence having at least 90% sequence identity with the nucleotide sequence of SEQ ID NO: 9; and/or the fourth probe comprises the nucleotide sequence of SEQ ID NO: 10, or comprises a nucleotide sequence having at least 90% sequence identity with the nucleotide sequence of SEQ ID NO: 10.

19. The method of claim 15, wherein the fifth probe comprises the nucleotide sequence of SEQ ID NO: 13, or comprises a nucleotide sequence having at least 90% sequence identity with the nucleotide sequence of SEQ ID NO: 13; and/or the sixth probe comprises the nucleotide sequence of SEQ ID NO: 14, or comprises a nucleotide sequence having at least 90% sequence identity with the nucleotide sequence of SEQ ID NO: 14.

20. The method of claim 1, wherein the aggregated difference of the melting temperature is determined based on an equation set forth as follows:
aggregated difference=\sqrt{Σᵢ₌₀ⁿ(Tmᵢ−Tmᵢ^{ref})²}, wherein Tmᵢ is a melting temperature determined for probe i, Tmᵢ^{ref} is a reference melting temperature determined for probe i, and n is an integer that denotes the number of probes.

21. The method of claim 1, wherein the one or more probes comprise one or more labels.

22. The method of claim 21, wherein the one or more labels comprise at least one of a fluorophore and a quencher.

23. The method of claim 22, wherein the fluorophore is selected from fluorescein, cyanine 3, cyanine 5, TexasRed, and TAMRA.

24. The method of claim 22, wherein the quencher is selected from BHQ1, BHQ2, and DABCYL.

25. An isolated nucleic acid for identifying a SARS-CoV-2 variant, comprising a nucleotide sequence of SEQ ID NO: 1-14 or comprising a nucleotide sequence having at least 90% sequence identity with a nucleotide sequence of SEQ ID NO: 1-14.

26. The isolated nucleic acid of claim 25, comprising a nucleotide sequence of SEQ ID NO: 5, 6, 9, 10, 13, and 14, or comprising a nucleotide sequence having at least 90% sequence identity with a nucleotide sequence of SEQ ID NO: 5, 6, 9, 10, 13, and 14.

27. A kit for identifying a SARS-CoV-2 variant, comprising the isolated nucleic acid of claim 25.


SEQUENCE LISTING (16 sequences)

SEQ ID NO: 1 - DNA, 27 bp, synthetic construct
  source 1..27: mol_type = other DNA
ggttttaattgttactttcctttacaa

SEQ ID NO: 2 - DNA, 27 bp, synthetic construct
  source 1..27: mol_type = other DNA
gaaagtactactactctgtatggttgg

SEQ ID NO: 3 - DNA, 39 bp, synthetic construct
  source 1..39: mol_type = other DNA
  misc_feature 7
  misc_feature 24: note = propynyl-dU
ccgcgttccatcccactaatgctgtggttaccaacgcgg

SEQ ID NO: 4 - DNA, 39 bp, synthetic construct
  source 1..39: mol_type = other DNA
  misc_feature 7: note = propynyl-dU
  misc_feature 24: note = propynyl-dU
cgcggttccatcccacttatgctgtggttaccaaccgcg

SEQ ID NO: 5 - DNA, 20 bp, synthetic construct
  source 1..20: mol_type = other DNA
  misc_feature 15: note = G as alternative, wobble primer
  misc_feature 18: note = C as alternative, wobble primer
ctatcaggccggtaacaaac

SEQ ID NO: 6 - DNA, 25 bp, synthetic construct
  source 1..25: mol_type = other DNA
  unsure 14: note = C as alternative, wobble primer
  misc_feature 6: note = T as alternative, wobble primer
gaaaccatatgattgtaaaggaaag

SEQ ID NO: 7 - DNA, 20 bp, synthetic construct
  source 1..20: mol_type = other DNA
ctatcaggccggtagcacac

SEQ ID NO: 8 - DNA, 25 bp, synthetic construct
  source 1..25: mol_type = other DNA
gaaaccatatgattgtaaaggaaag

SEQ ID NO: 9 - DNA, 44 bp, synthetic construct
  source 1..44: mol_type = other DNA
ccgcgccttgtaatggtgttgaaggttttaattgttacgcgcgg

SEQ ID NO: 10 - DNA, 44 bp, synthetic construct
  source 1..44: mol_type = other DNA
ccgcgccttgtaatggtgttaaaggttttaattgttacgcgcgg

SEQ ID NO: 11 - DNA, 20 bp, synthetic construct
  source 1..20: mol_type = other DNA
aggctgcgttatagcttgga

SEQ ID NO: 12 - DNA, 26 bp, synthetic construct
  source 1..26: mol_type = other DNA
cagttgaaatatctctctcaaaaggt

SEQ ID NO: 13 - DNA, 46 bp, synthetic construct
  source 1..46: mol_type = other DNA
ccgcgtaattataattacctgtatagattgtttaggaagtccgcgg

SEQ ID NO: 14 - DNA, 48 bp, synthetic construct
  source 1..48: mol_type = other DNA
cgccgaggtggtaattataattaccggtatagattgtttaggtcggcg

SEQ ID NO: 15 - DNA, 20 bp, synthetic construct
  source 1..20: mol_type = other DNA
aggctgcgttatagcttgga

SEQ ID NO: 16 - DNA, 20 bp, synthetic construct
  source 1..20: mol_type = other DNA
aaacagttgctggtgcatgt